(19) United States
(12) Patent Application Publication
(10) Pub. No.: US 2022/0354825 A1
(43) Pub. Date: Nov. 10, 2022

(54) PHARMACEUTICAL COMPOSITION FOR PREVENTION OR TREATMENT OF INFLAMMATORY DISEASES COMPRISING NAPHTHOQUINONE DERIVATIVE

(71) Applicant: LMITO THERAPEUTICS INC., Seongnam-si, Gyeonggi-do (KR)

(72) Inventors: Whee Seong LEE, Seongnam-Si, Gyeonggi-do (KR); Eun Ju LEE, Suwon-si, Gyeonggi-do (KR); In Seok KO, Pyoungtaek-si, Gyeonggi-do (KR)

(73) Assignee: LMITO THERAPEUTICS INC., Seongnam-si, Gyeonggi-do (KR)

(21) Appl. No.: 17/595,745

(22) PCT Filed: Jun. 1, 2020
(86) PCT No.: PCT/KR2020/007099

§ 371 Date: Nov. 23, 2021

(30) Foreign Application Priority Data
May 31, 2019 (KR) 10-2019-0064422

(51) Int. Cl. A61K 31/416; A61P 1/04; A61P 1/00; A61K 31/4192; A61K 31/4184; A61K 31/5377; A61K 31/423; A61K 31/428; A61K 31/454

(52) U.S. Cl. CPC A61K 31/416; A61P 1/04; A61P 1/00; A61K 31/4192; A61K 31/4184; A61K 31/5377; A61K 31/423; A61K 31/428; A61K 31/454

(57) ABSTRACT

Provided is a method for the prevention or treatment of inflammatory diseases using a naphthoquinone or benzoindazole compound which increases the ratio of NAD⁺ and NAD⁺/NADH through activity in NQO1 in vivo, and through this, activates mitochondria, thereby inducing the metabolism of macrophages towards mitochondrial OXPHOS, which is the major metabolic pathway of M2 phenotype macrophages, such that the macrophages are polarized into an anti-inflammatory macrophage M2 phenotype, and consequently is able to inhibit the expression and activity of inflammatory cytokines, or a pharmaceutically acceptable salt, hydrate, solvate, enantiomer, diasteromer, tautomer, or prodrug thereof.

### TECHNICAL FILED

The present invention relates to a pharmaceutical composition for the prevention or treatment of an inflammatory disease comprising a naphthoquinone derivative.

### BACKGROUND ART

Our body's immune system has various defense systems which are able to protect the host from internal stimuli or exterior pathogens. It is very important to properly balance the immune system in maintaining health, and it is called as an immune homeostasis. Immune action to maintain immune homeostasis can be divided into immune response enhancing immunity, and immune tolerance suppressing excessive increase of the immune response.

The imbalance of immune homeostasis can be caused by various internal or external factors of the human body. If immune response is stronger than the immune tolerance, that is, when the immune cells are excessively activated, inflammatory or autoimmune diseases may occur.

Conversely, if immune tolerance is stronger than the immune response, that is, when the immune system does not working its function properly, diseases such as infectious diseases or cancer occur.

Accordingly, attempts are being progressed to treat various immune-related diseases by maintaining immune homeostasis that balances between activation and suppression of immune response.

A typical disease caused by excessive immune response is inflammatory disease. Among the inflammatory diseases, ulcerative colitis is an inflammatory bowel disease in which inflammation or ulcers occur in the colon due to genetic factors or excessive immune response. Common symptoms include severe abdominal pain, weight loss, and bleeding along with feces or diarrhea containing blood and mucus. In many cases, exacerbation and improvement may be repeated, and other complications or colon cancer may be progressed. Although researches relating to ulcerative colitis have been actively conducted, no treatment for complete curing has been developed. Accordingly, anti-inflammatory drugs or adrenocortical hormones have been commonly used in practice, and immune-suppressants, steroids, antibiotics or the like have sometimes been used depending on the patient's conditions. Although complete cure is possible through surgery, drug therapy has been recommended rather than surgery, due to the complexity of the surgery or the high side effects caused by sequela of the surgery.

In order to reduce excessive immune responses and maintain immune homeostasis, there are various types of immune suppressor cells in the human body, among which macrophages are very important immune cells responsible for innate immunity and are distributed in various forms in all tissues of the our body.

Macrophages protect the body by destroying the antigens through toxins secreting and phagocytosis when external antigens invade in normal conditions. Also, macrophages have a wide variety of roles in immune responses, such as wound healing and inflammatory reactions in our bodies. Macrophages are traditionally divided into M1 or M2 phenotypes by pathological conditions. However, according to recent research trends, it is known that macrophage has various forms of phenotype depending on the origin, place, microenvironment, and pathological situation of macrophages rather than this traditional dichotomy classification. M1 phenotypic pro-inflammatory macrophage is activated by lipopolysaccharides (LPS) or tumor necrosis factor α (INF-α), and secretes IL-1β, IL-6, and INF-α, the major metabolic pathway is glycolysis in the cytosol rather than metabolism of mitochondria. On the other hand, the main metabolic pathway of M2 phenotypic anti-inflammatory macrophage is mitochondrial oxidative phosphorylation (OXPHOS), it is activated by IL-4 or IL-10, and it plays an important role in mitigation of inflammation or wounds healing. Therefore, polarization of macrophages from M1 phenotype to M2 phenotype enables inflammation relief or wound healing.

### DETAILED DESCRIPTION OF THE INVENTION

### Technical Problem to be Solved

An object of the present invention is to provide a pharmaceutical composition that can be used to prevent or treat inflammatory diseases by inhibiting the expression and activity of inflammatory cytokines through converting from glycolysis metabolism of pro-inflammatory M1-type macrophage to mitochondrial oxidative phosphorylation (OXPHOS) which can polarize to anti-inflammatory M2-type macrophage.

Another object of the present invention is to provide a method for preventing or treating inflammatory diseases by inhibiting the expression and activity of inflammatory cytokines through converting from glycolysis metabolism of pro-inflammatory M1-type macrophage to mitochondrial oxidative phosphorylation (OXPHOS) which can polarize to anti-inflammatory M2-type macrophage.

### Technical Solution

The inventors have found that a naphthoquinone compound represented by the following Formula 1 or a pharmaceutically acceptable salt can react with an NQO1 enzyme to lead to mitochondrial activation through increasing NAD+/NADH ratio, by which macrophage metabolism can be induced into the direction of mitochondrial OXPHOS which is the main metabolic path of M2 phenotype, rather than glycolysis of M1 phenotype in cytosol, so as to polarize the macrophages to anti-inflammatory M2 type macrophages, resulting in the inhibition of the expression and activity of inflammatory cytokines. Therefore, the technical solution of the present invention to solve the technical problem is as follows:

1. A pharmaceutical composition for preventing or treating an inflammatory disease comprising a compound represented by the following Formula 1, or a pharmaceutically acceptable salt, a hydrate, solvate, an enantiomer, a diasteromer, a tautomer or a prodrug thereof:

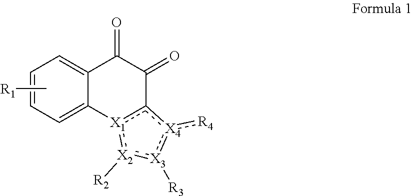

wherein,

X₁, X₂, X₃ and X₄ are each independently selected from the group consisting of carbon, nitrogen, oxygen and sulfur atoms, wherein at least two of X₁, X₂, X₃ and X₄ are hetero atoms selected from nitrogen, oxygen and sulfur, provided that X₁ and X₄ cannot simultaneously be nitrogen;

R₁ is one or more selected from the group consisting of H, alkyl, alkyloxy, C₆₋₁₀ aryl, heteroaryl, halo, nitro, hydroxy, cyano and —NR₅R₆;

R₂ is not present, or selected from the group consisting of H, O, alkyl, alkyloxy, C₆₋₁₀ aryl and heterocyclyl, wherein the alkyl may be substituted with C₆₋₁₀ aryl, and the heterocyclyl may be substituted with —C(O)R₈;

R₃ is not present, or selected from the group consisting of H, O, halo, alkyl, alkyloxy, C₆₋₁₀ aryl, heterocyclyl, —SO₂NR₇R₁₂, —NR₉R₁₀ and —C(O)R₁₁, wherein when the alkyl is substituted, its substituent is selected from the group consisting of halo, alkyloxy, C₆₋₁₀ aryl, C₆₋₁₀ aryloxy, heterocyclyl, —C(O)R₈, R₁₂C(O)O— and —NR₁₃R₁₄, and the heterocyclyl may be substituted with —C(O)R₈;

R₄ is not present, or selected from the group consisting of H, O, alkyl, alkyloxy, C₆₋₁₀ aryl, C₆₋₁₀ aryloxy, heterocyclyl and —C(O)R₁₅, wherein when the alkyl is substituted, its substituent is selected from the group consisting of halo, C₆₋₁₀ aryl, heterocyclyl and —C(O)R₈, and the heterocyclyl may be substituted with —C(O)R₈;

R₅ and R₆ are each independently selected from the group consisting of H, alkyl and —C(O)R₇, or R₅ and R₆ are joined with each other to form a heterocyclyl including at least one nitrogen atom in the cycle;

R₇ and R₁₂ are each alkyl, or R₇ and R₁₂ are joined with each other to form a heterocyclyl including at least one nitrogen atom in the cycle;

R₁₁ is heterocyclyl or —NR₁₃R₁₄;

R₁₅ is alkyl, alkyloxy, C₆₋₁₀ aryloxy, heterocyclyl or —NR₁₃R₁₄;

R₉, R₁₀, R₁₃ and R₁₄ are each independently selected from the group consisting of H, alkyl, unsubstituted or halo-substituted C₆₋₁₀ aryl, and —C(O)R₈, or either R₉ and R₁₀ are jointed with each other, or R₁₃ and R₁₄ are jointed with each other, to form a heterocyclyl including at least one nitrogen atom in the cycle;

R₈ is alkyloxy;

the alkyl is each C₆₋₁₀ linear or branched alkyl, or C₃₋₇ cyclic alkyl, the heterocyclyl is 3- to 7-membered heterocyclic group having in the cycle at least one hetero atom selected from the group consisting of N, O and S, the heteroaryl is 5- to 10-membered aromatic cyclic group having in the cycle at least one hetero atom selected from N, O and S, and when the aryl or heteroaryl is substituted, its substituent is each at least one selected from the group consisting of halo, alkyl, halo-substituted alkyl and alkyloxy; and

[symbol] is a single bond or a double bond depending on R₂, R₃, R₄, X₁, X₂, X₃ and X₄,

provided that when both X₁ and X₄ are carbon atom, and both X₂ and X₃ are nitrogen atom, either of R₂ or R₄ is not alkyl, aryl or heterocyclyl, and herein, when R₂ is alkyl, aryl or heterocyclyl, R₄ is not —C(O)R₁₅; and when both X₁ and X₄ are carbon atom, and both X₃ and X₄ are nitrogen atom, either of R₂ or R₄ is O or alkyloxy.

### Advantageous Effects

According to the prevent invention, a pharmaceutical composition for preventing or treating inflammatory diseases comprising a naphthoquinone or benzoindazolone compound, or a pharmaceutically acceptable salt, hydrate, solvate, enantiomer, diastereomer, tautomer or prodrug thereof, which can increase NAD⁺ and NAD⁺/NADH ratio by in vivo NQO1 activity, thereby inhibiting the expression and activity of inflammatory cytokines, was provided.

In the present invention, it was found that a naphthoquinone or benzoindazolone compound acts on NQO1 enzyme in the cytosol of macrophage so as to increase NAD+ and activate Sirtuin, by which mitochondrial function was improved. Inflammatory M1 type macrophages obtain energy mainly through glycolysis in the cytosol because the enzymes acting to TCA cycle of mitochondria are not fully working. However, the compound of the present invention improves the function of mitochondria such that the main metabolic path changes from glycolysis in the cytosol to OXPHOS of mitochondria. In other words, the compound of the present invention induces polarization to M2 type macrophages, so that can suppress the expression and activity of inflammatory cytokines, and thus, it can be useful in the treatment of inflammatory diseases.

### BRIEF DESCRIPTION OF DRAWINGS

FIGS. 1 to 5 show NAD+ changes in the macrophages when treated with Compounds 6, 16, 17, 22 and 27, respectively (in order from left in each figure: Untreatment, LPS/ATP, LPS/ATP+1 μM compound, LPS/ATP+5 μM compound, and LPS/ATP+10 μM compound).

FIGS. 6 to 10 show NAD+/NADH changes in the macrophage when treated with Compounds 6, 16, 17, 22 and 27, respectively (in order from left in each figure: Untreatment, LPS/ATP, LPS/ATP+1 μM compound, LPS/ATP+5 μM compound, and LPS/ATP+10 μM compound).

FIGS. 11 to 12 show mitochondrial reactive oxygen species changes in the macrophage when treated with Compounds 6 and 27, respectively (in order from left in each figure: Untreatment, LPS/ATP, LPS/ATP+1 μM compound, LPS/ATP+5 μM compound, and LPS/ATP+10 μM compound).

FIGS. 13 to 17 show ATP increases in the macrophage when treated with Compounds 6, 16, 17, 22 and 27, respectively (in order from left in each figure: Untreatment, LPS+SC, LPS+1 μM compound, LPS+5 μM compound, and LPS+10 μM compound).

FIG. 18 shows the mitochondrial oxygen consumption rates in the macrophage when treated with Compounds 6 and 27, respectively.

FIG. 19 shows survival rates in the inflammatory bowel disease mouse model when treated with Compounds 6 and 27, respectively.

FIG. 20 shows body weight changes in the inflammatory bowel disease mouse model when treated with Compounds 6 and 27, respectively.

FIG. 21 shows colitis score changes in the inflammatory bowel disease mouse model when treated with Compounds 6 and 27, respectively (in order from left in the figure, respectively: DSS, DSS+Compound 27, and DSS+Compound 6).

FIG. 22 shows cytokine secretions in the inflammatory bowel disease mouse model when treated with Compounds 6 and 27, respectively (in order from left in the figure, respectively: DSS, DSS+Compound 27, and DSS+Compound 6).

FIG. 23 shows MPO activities in the inflammatory bowel disease mouse model when treated with Compounds 6 and 27, respectively (in order from left in the figure, respectively: DSS, DSS+Compound 27, and DSS+Compound 6).

FIG. 24 shows SIRT expressions in the inflammatory bowel disease mouse model when treated with Compounds 6 and 27, respectively.

FIG. 25 shows immunohistochemistry results in the inflammatory bowel disease mouse model when treated with Compounds 6 and 27, respectively.

FIG. 26 shows histopathological scores in the inflammatory bowel disease mouse model when treated with Compounds 6 and 27, respectively.

### DETAILED DESCRIPTION OF THE EMBODIMENTS

### Definition of Terms

The terms used in the present disclosure are briefly defined herein.

The term “pharmaceutically acceptable salt” means a form of a compound which does not cause any serious stimuli in an organism to which the compound is administered, and does not destroy biological activities and physical properties of the compound.

The terms “hydrate”, “solvate”, “prodrug”, “tautomer”, “enantiomer” and “diastereomer” also mean forms of a compound which does not cause any serious stimuli in an organism to which the compound is administered, and does not destroy biological activities and physical properties of the compound.

The pharmaceutically acceptable salt includes an acid-adduct salt which is formed by addition of an inorganic acid, such as hydrochloric acid, sulfuric acid, nitric acid, phosphoric acid, hydrobromic acid, hydriodic acid and the like, or an organic acid, such as tartaric acid, formic acid, citric acid, acetic acid, trichloroacetic acid, fluoroacetic acid, gluconic acid, benzoic acid, lactic acid, fumaric acid, maleic acid, salicylic acid, methanesulfonic acid, ethanesulfonic acid, benzenesulfonic acid, p-toluenesulfonic acid and the like. In case that a carboxyl acid group is present in the compound of Formula 1 above, an example of a pharmaceutically acceptable carboxylic acid salt includes a metal salt or an alkaline earth metal salt formed with lithium, sodium, potassium, calcium, magnesium or the like; an amino acid salt formed with lysine, arginine, guanidine or the like; and an organic salt formed with dicyclohexylamine, N-methyl-D-glucamine, tris(hydroxymethyl)methylamine, diethanolamine, choline, trimethylamine or the like. The compound of Formula 1 according to the present invention may be converted into its salt by a conventional method.

The term “hydrate” means a compound according to the present invention containing a stoichiometric or non-stoichiometric amount of water bound through non-covalent intermolecular forces, or a salt thereof.

The term “solvate” means a compound according to the present invention containing a stoichiometric or non-stoichiometric amount of solvent bound through non-covalent intermolecular forces, or a salt thereof. A solvent for the solvate may be any solvent which is volatile, non-toxic and/or suitable for administration to a human.

The term “prodrug” means a substance which can be converted in vivo into the compound of Formula 1 according to the present invention. In some cases, a prodrug is often used because it may be more easily administered than its parent drug. For example, biological activities can be achieved by oral administration of a prodrug, while it is not possible with its parent drug. In addition, a prodrug may have better solubility compared with its parent drug in a pharmaceutical formulation. For example, a prodrug may be in the form of an ester (a “prodrug”), which is easy to pass through cell membrane and can be hydrolyzed by a metabolism into a carboxylic acid as an active form within a cell where its water solubility is beneficial, although its water solubility is disadvantageous for transportation. Another example of the prodrug may be a short peptide (a poly-amino acid), in which a peptide is linked to an acid group, which is metabolized so that its active site is exposed.

The term “tautomer” means a type of structural isomers having an identical chemical or molecular formula, but different coupling between constituent atoms. For example, its structure is converted into each other between both isomers, such as a keto-enol structure.

The term “enantiomer” or “diastereomer” means an isomer which occurs due to different arrangements of atoms in a molecule even having an identical Formula or molecular formula. The term “enantiomer” means an isomer which is not superimposed with its mirror image, like a relation between a right hand and a left hand. In addition, the term “diastereomer” means a stereoisomer which is not in a mirror image relation. All isomers and mixtures thereof are also within the scope of the present invention.

The term “alkyl” means an aliphatic hydrocarbyl group, which includes “saturated alkyl,” and “unsaturated alkyl” containing at least one double bond or triple bond, and includes a C₁₋₁₀ linear and branched alkyl, and C₃₋₇ cyclic alkyl.

The term “aryl” means a C₁₋₁₀ aromatic cyclic group, and the term “heterocyclyl” means a 3- to 7-membered cyclic group having at least one hetero atom selected from the group consisting of nitrogen, oxygen and sulfur in the cycle, and the term “heteroaryl” means a 5- to 10-membered hetero aromatic cyclic group having at least one hetero atom selected from the group consisting of nitrogen (N), oxygen (O) and sulfur (S) in the cycle.

Hereinafter, the present invention will be described in more detail.

In one embodiment of the present invention, the compound represented by Formula 1 may be a compound wherein X₁ and X₄ are carbon atoms, and X₂ and X₃ are nitrogen atoms. Herein, R₂ of Formula 1 is not present, or alkyl, alkyloxy or C₆₋₁₀ aryl, R₃ is not present, or H, alkyl or C₆₋₁₀ aryl, and R₄ is not present, or O, alkyl or alkyloxy, provided that either R₂ or R₄ is not alkyl or aryl, wherein when the alkyl or aryl is substituted, its substituents are as defined above.

In another embodiment of the present invention, the compound of Formula 1 may be a compound wherein X₁ and X₃ are carbon atoms, and X₂ and X₄ are nitrogen atoms. Herein, R₂ of Formula 1 is not present or alkyl, R₃ is selected from the group consisting of halo, alkyl, alkyloxy, C₆₋₁₀ aryl and heterocyclyl, and R₄ is not present, or may be selected from the group consisting of H, alkyl, C₆₋₁₀ aryl, C₆₋₁₀ aryloxy, heterocyclyl and —C(O)R₁₅. Herein, R₁₅, and substituents of alkyl, aryl and heterocyclyl, which may be substituted, are as defined above.

In another embodiment of the present invention, the compound of Formula 1 may be a compound wherein X₁ and X₃ are carbon atoms, X₂ is nitrogen atom, and X₄ is sulfur atom. Herein, R₂ and R₄ of Formula 1 are not present, and R₃ is alkyl or C₆₋₁₀ aryl, wherein when the alkyl and aryl are substituted, its substituents are as defined above.

In another embodiment of the present invention, the compound of Formula 1 may be a compound wherein X₁ and X₃ are carbon atoms, one of X₂ and X₄ is nitrogen atom and the other is oxygen atom. Herein, R₂ of Formula 1 is not present, R₃ is O, alkyl or C₆₋₁₀ aryl, and R₄ is not present, or H or alkyl, wherein when the alkyl and aryl are substituted, its substituents are as defined above.

In another embodiment of the present invention, the compound of Formula 1 may be a compound wherein X₂, X₃ and X₄ are nitrogen atoms. Herein, R₂ of Formula 1 is not present, or alkyl or heterocyclyl, R₃ is not present, or may be selected from the group consisting of alkyl, C₆₋₁₀ aryl, heterocyclyl, —SO₂R₇, —NR₉R₁₀ and —C(O)R₁₁, and R₄ is not present, or may be selected from the group consisting of alkyl, heterocyclyl and —C(O)R₁₅. Herein, R₇, R₉, R₁₀, R₁₁ and R₁₅, and substituents of alkyl, aryl and heterocyclyl, which may be substituted, are as defined above.

In another embodiment of the present invention, the compound of Formula 1 may be a compound wherein X₂ and X₃ are carbon atoms, X₁ and X₄ are nitrogen atoms. Herein, R₂ of Formula 1 is C₆₋₁₀ aryl, and R₃ and R₄ are not present, wherein when the aryl is substituted, its substituents are as defined above.

In the compound of Formula 1, the halo is any one of fluoro, chloro, bromo and iodo, the aryl is preferably pheny, and the heteroaryl is 5- to 10-membered heteroaromatic cyclic group, examples of which may be pyridinyl, pyridazinyl, pyrrolyl, pyrazolyl, imidazoly, oxazoly, thiazolyl, furanyl, etc., but not limited thereto. The heterocyclyl is 3- to 7-membered aliphatic heterocyclic group having in the ring at least hetero atom selected from N, O and S, examples of which may be aziridinyl, azetidinyl, pyrrolidinyl, piperidinyl, piperazinyl, morpholinyl, etc., but not limited thereof.

In the present invention, the inflammatory disease includes ulcerative colitis, sepsis, rheumatoid arthritis, multiple sclerosis, Crohn's disease, atopic dermatitis, etc., but not limited thereto, and it may include any inflammatory disease which can be prevented or treated by inhibiting the expression and activity of inflammatory cytokines through increasing NAD+ and NAD⁺/NADH ratio with NQO1 activity.

In the present invention, the specific embodiments of the compound of Formula 1 include the compounds as shown in Table 1 below:

TABLE 1

| Nos. | Compounds |
|---|---|
| 1 | 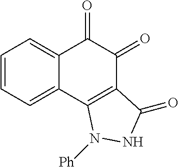 [SMILES: O=C1C(=O)c2c(n(-c3ccccc3)[nH]c2=O)-c2ccccc21] |
| 2 | 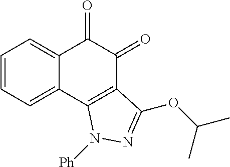 [SMILES: CC(C)Oc1nn(-c2ccccc2)c2c1C(=O)C(=O)c1ccccc1-2] |
| 3 | 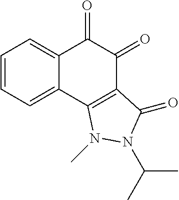 [SMILES: CC(C)n1c(=O)c2c(n1C)-c1ccccc1C(=O)C2=O] |
| 4 | 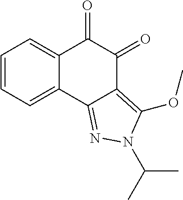 [SMILES: COc1c2c(nn1C(C)C)-c1ccccc1C(=O)C2=O] |
| 5 | 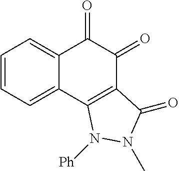 [SMILES: Cn1c(=O)c2c(n1-c1ccccc1)-c1ccccc1C(=O)C2=O] |
| 6 | 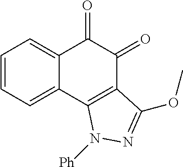 [SMILES: COc1nn(-c2ccccc2)c2c1C(=O)C(=O)c1ccccc1-2] |
| 7 | 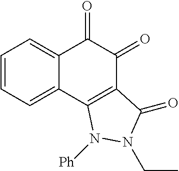 [SMILES: CCn1c(=O)c2c(n1-c1ccccc1)-c1ccccc1C(=O)C2=O] |
| 8 | 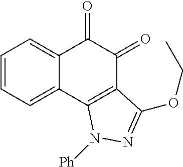 [SMILES: CCOc1nn(-c2ccccc2)c2c1C(=O)C(=O)c1ccccc1-2] |
| 9 | 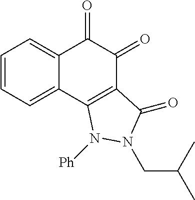 [SMILES: CC(C)Cn1c(=O)c2c(n1-c1ccccc1)-c1ccccc1C(=O)C2=O] |
| 10 | 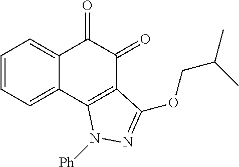 [SMILES: CC(C)COc1nn(-c2ccccc2)c2c1C(=O)C(=O)c1ccccc1-2] |
| 11 | 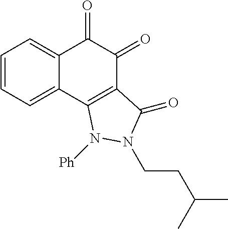 [SMILES: CC(C)CCn1c(=O)c2c(n1-c1ccccc1)-c1ccccc1C(=O)C2=O] |
| 12 | 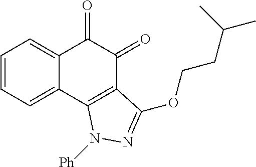 [SMILES: CC(C)CCOc1nn(-c2ccccc2)c2c1C(=O)C(=O)c1ccccc1-2] |
| 13 | 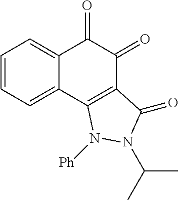 [SMILES: CC(C)n1c(=O)c2c(n1-c1ccccc1)-c1ccccc1C(=O)C2=O] |
| 14 | 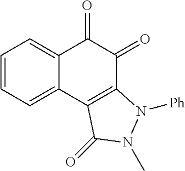 [SMILES: Cn1c(=O)c2c(n1-c1ccccc1)C(=O)C(=O)c1ccccc1-2] |
| 15 | 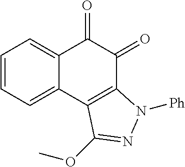 [SMILES: COc1nn(-c2ccccc2)c2c1-c1ccccc1C(=O)C2=O] |
| 16 | 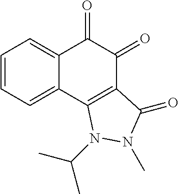 [SMILES: CC(C)n1c2c(c(=O)n1C)C(=O)C(=O)c1ccccc1-2] |
| 17 | 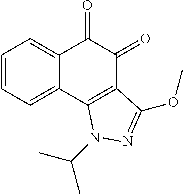 [SMILES: COc1nn(C(C)C)c2c1C(=O)C(=O)c1ccccc1-2] |
| 18 | 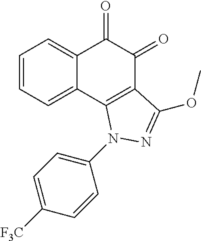 [SMILES: COc1nn(-c2ccc(C(F)(F)F)cc2)c2c1C(=O)C(=O)c1ccccc1-2] |
| 19 | 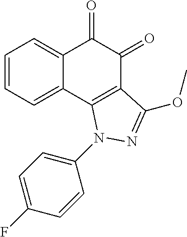 [SMILES: COc1nn(-c2ccc(F)cc2)c2c1C(=O)C(=O)c1ccccc1-2] |
| 20 | 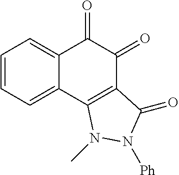 [SMILES: Cn1c2c(c(=O)n1-c1ccccc1)C(=O)C(=O)c1ccccc1-2] |
| 21 | 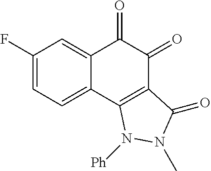 [SMILES: Cn1c(=O)c2c(n1-c1ccccc1)-c1ccc(F)cc1C(=O)C2=O] |
| 22 | 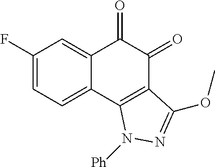 [SMILES: COc1nn(-c2ccccc2)c2c1C(=O)C(=O)c1cc(F)ccc1-2] |
| 23 | 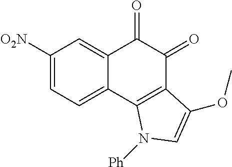 [SMILES: COc1cn(-c2ccccc2)c2c1C(=O)C(=O)c1cc([N+](=O)[O-])ccc1-2] |
| 24 | 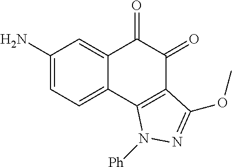 [SMILES: COc1nn(-c2ccccc2)c2c1C(=O)C(=O)c1cc(N)ccc1-2] |
| 25 | 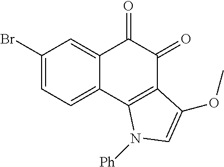 [SMILES: COc1cn(-c2ccccc2)c2c1C(=O)C(=O)c1cc(Br)ccc1-2] |
| 26 | 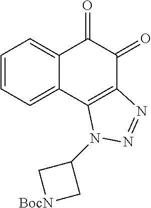 [SMILES: CC(C)(C)OC(=O)N1CC(n2nnc3c2-c2ccccc2C(=O)C3=O)C1] |
| 27 | 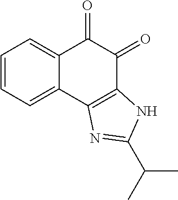 [SMILES: CC(C)c1nc2c([nH]1)C(=O)C(=O)c1ccccc1-2] |
| 28 | 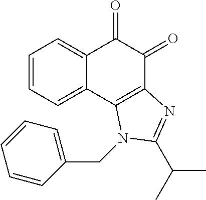 [SMILES: CC(C)c1nc2c(n1Cc1ccccc1)-c1ccccc1C(=O)C2=O] |
| 29 | 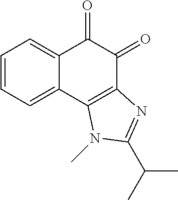 [SMILES: CC(C)c1nc2c(n1C)-c1ccccc1C(=O)C2=O] |
| 30 | 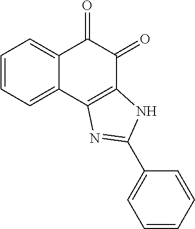 [SMILES: O=C1C(=O)c2[nH]c(-c3ccccc3)nc2-c2ccccc21] |
| 31 | 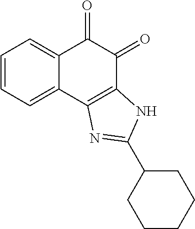 [SMILES: O=C1C(=O)c2[nH]c(C3CCCCC3)nc2-c2ccccc21] |
| 32 | 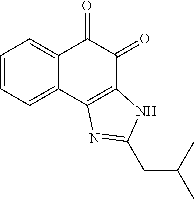 [SMILES: CC(C)Cc1nc2c([nH]1)C(=O)C(=O)c1ccccc1-2] |
| 33 | 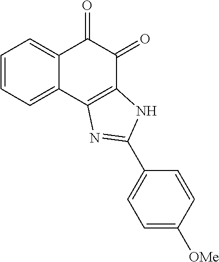 [SMILES: COc1ccc(-c2nc3c([nH]2)C(=O)C(=O)c2ccccc2-3)cc1] |
| 34 | 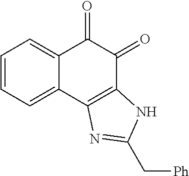 [SMILES: O=C1C(=O)c2[nH]c(Cc3ccccc3)nc2-c2ccccc21] |
| 35 | 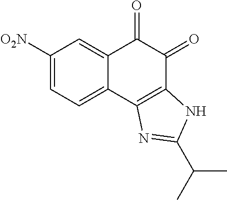 [SMILES: CC(C)c1nc2c([nH]1)C(=O)C(=O)c1cc([N+](=O)[O-])ccc1-2] |
| 36 | 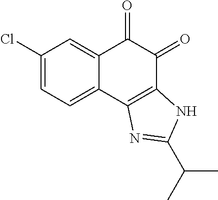 [SMILES: CC(C)c1nc2c([nH]1)C(=O)C(=O)c1cc(Cl)ccc1-2] |
| 37 | 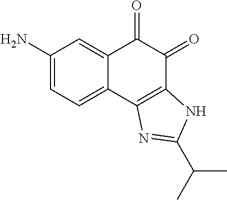 [SMILES: CC(C)c1nc2c([nH]1)C(=O)C(=O)c1cc(N)ccc1-2] |
| 38 | 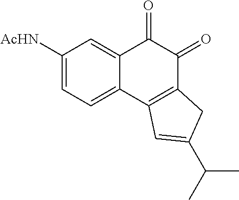 [SMILES: CC(=O)Nc1ccc2c(c1)C(=O)C(=O)C1=C2C=C(C(C)C)C1] |
| 39 | 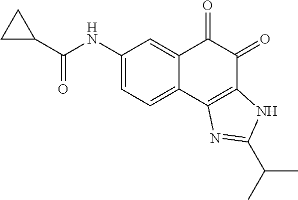 [SMILES: CC(C)c1nc2c([nH]1)C(=O)C(=O)c1cc(NC(=O)C3CC3)ccc1-2] |
| 40 | 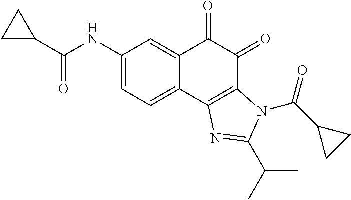 [SMILES: CC(C)c1nc2c(n1C(=O)C1CC1)C(=O)C(=O)c1cc(NC(=O)C3CC3)ccc1-2] |
| 41 | 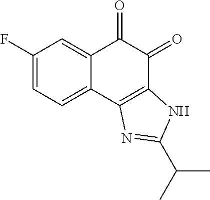 [SMILES: CC(C)c1nc2c([nH]1)C(=O)C(=O)c1cc(F)ccc1-2] |
| 42 | 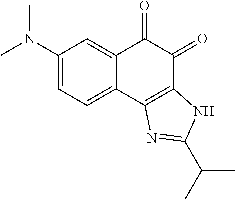 [SMILES: CC(C)c1nc2c([nH]1)C(=O)C(=O)c1cc(N(C)C)ccc1-2] |
| 43 | 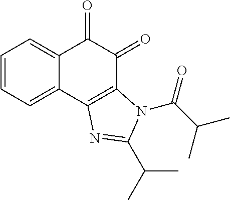 [SMILES: CC(C)C(=O)n1c(C(C)C)nc2c1C(=O)C(=O)c1ccccc1-2] |
| 44 | 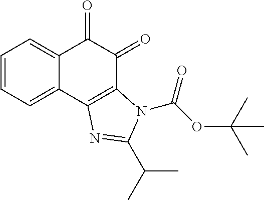 [SMILES: CC(C)c1nc2c(n1C(=O)OC(C)(C)C)C(=O)C(=O)c1ccccc1-2] |
| 45 | 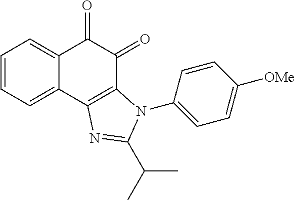 [SMILES: COc1ccc(-n2c(C(C)C)nc3c2C(=O)C(=O)c2ccccc2-3)cc1] |
| 46 | 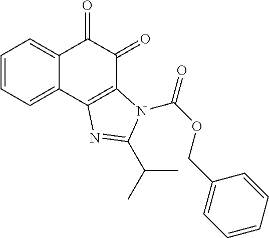 [SMILES: CC(C)c1nc2c(n1C(=O)OCc1ccccc1)C(=O)C(=O)c1ccccc1-2] |
| 47 | 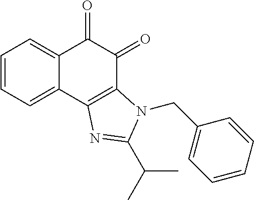 [SMILES: CC(C)c1nc2c(n1Cc1ccccc1)C(=O)C(=O)c1ccccc1-2] |
| 48 | 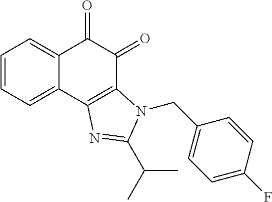 [SMILES: CC(C)c1nc2c(n1Cc1ccc(F)cc1)C(=O)C(=O)c1ccccc1-2] |
| 49 | 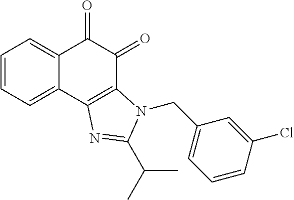 [SMILES: CC(C)c1nc2c(n1Cc1cccc(Cl)c1)C(=O)C(=O)c1ccccc1-2] |
| 50 | 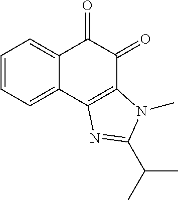 [SMILES: CC(C)c1nc2c(n1C)C(=O)C(=O)c1ccccc1-2] |
| 51 | 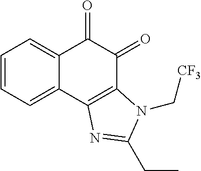 [SMILES: CCc1nc2c(n1CC(F)(F)F)C(=O)C(=O)c1ccccc1-2] |
| 52 | 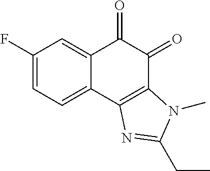 [SMILES: CCc1nc2c(n1C)C(=O)C(=O)c1cc(F)ccc1-2] |
| 53 | 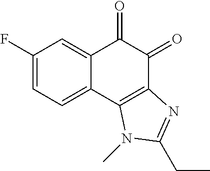 [SMILES: CCc1nc2c(n1C)-c1ccc(F)cc1C(=O)C2=O] |
| 54 | 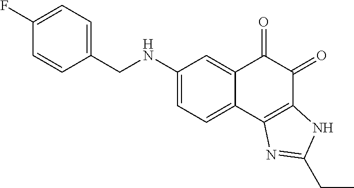 [SMILES: CCc1nc2c([nH]1)C(=O)C(=O)c1cc(NCc3ccc(F)cc3)ccc1-2] |
| 55 | 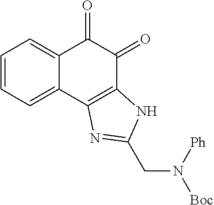 [SMILES: CC(C)(C)OC(=O)N(Cc1nc2c([nH]1)C(=O)C(=O)c1ccccc1-2)c1ccccc1] |
| 56 | 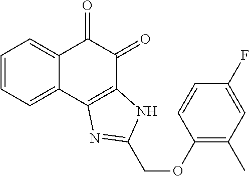 [SMILES: Cc1cc(F)ccc1OCc1nc2c([nH]1)C(=O)C(=O)c1ccccc1-2] |
| 57 | 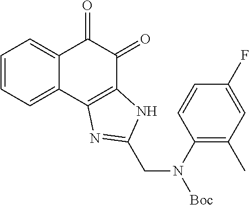 [SMILES: Cc1cc(F)ccc1N(Cc1nc2c([nH]1)C(=O)C(=O)c1ccccc1-2)C(=O)OC(C)(C)C] |
| 58 | 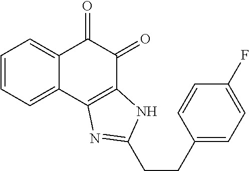 [SMILES: O=C1C(=O)c2[nH]c(CCc3ccc(F)cc3)nc2-c2ccccc21] |
| 59 | 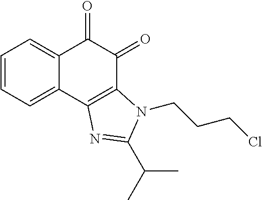 [SMILES: CC(C)c1nc2c(n1CCCCl)C(=O)C(=O)c1ccccc1-2] |
| 60 | 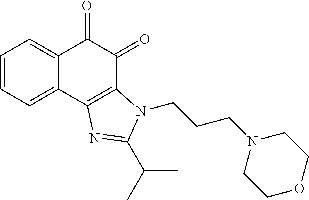 [SMILES: CC(C)c1nc2c(n1CCCN1CCOCC1)C(=O)C(=O)c1ccccc1-2] |
| 61 | 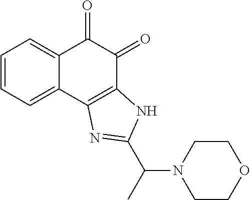 [SMILES: CC(c1nc2c([nH]1)C(=O)C(=O)c1ccccc1-2)N1CCOCC1] |
| 62 | 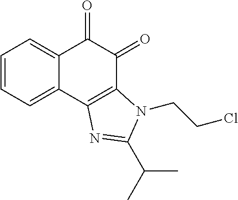 [SMILES: CC(C)c1nc2c(n1CCCl)C(=O)C(=O)c1ccccc1-2] |
| 63 | 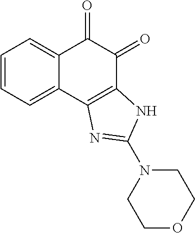 [SMILES: O=C1C(=O)c2[nH]c(N3CCOCC3)nc2-c2ccccc21] |
| 64 | 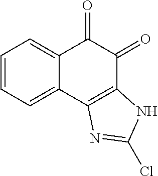 [SMILES: O=C1C(=O)c2[nH]c(Cl)nc2-c2ccccc21] |
| 65 | 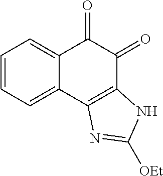 [SMILES: CCOc1nc2c([nH]1)C(=O)C(=O)c1ccccc1-2] |
| 66 | 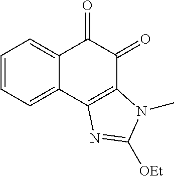 [SMILES: CCOc1nc2c(n1C)C(=O)C(=O)c1ccccc1-2] |
| 67 | 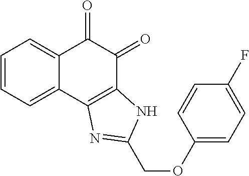 [SMILES: O=C1C(=O)c2[nH]c(COc3ccc(F)cc3)nc2-c2ccccc21] |
| 68 | 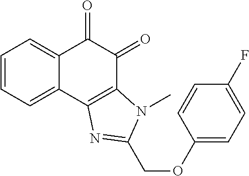 [SMILES: Cn1c(COc2ccc(F)cc2)nc2c1C(=O)C(=O)c1ccccc1-2] |
| 69 | 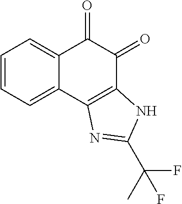 [SMILES: CC(F)(F)c1nc2c([nH]1)C(=O)C(=O)c1ccccc1-2] |
| 70 | 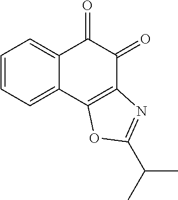 [SMILES: CC(C)c1nc2c(o1)-c1ccccc1C(=O)C2=O] |
| 71 | 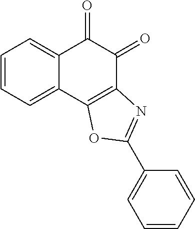 [SMILES: O=C1C(=O)c2nc(-c3ccccc3)oc2-c2ccccc21] |
| 72 | 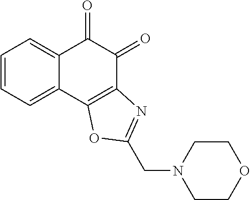 [SMILES: O=C1C(=O)c2nc(CN3CCOCC3)oc2-c2ccccc21] |
| 73 | 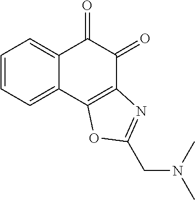 [SMILES: CN(C)Cc1nc2c(o1)-c1ccccc1C(=O)C2=O] |
| 74 | 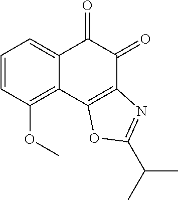 [SMILES: COc1cccc2c1-c1oc(C(C)C)nc1C(=O)C2=O] |
| 75 | 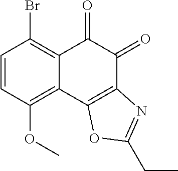 [SMILES: CCc1nc2c(o1)-c1c(OC)ccc(Br)c1C(=O)C2=O] |
| 76 | 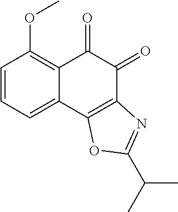 [SMILES: COc1cccc2c1C(=O)C(=O)c1nc(C(C)C)oc1-2] |
| 77 | 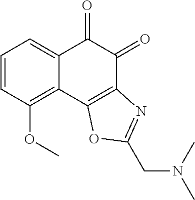 [SMILES: COc1cccc2c1-c1oc(CN(C)C)nc1C(=O)C2=O] |
| 78 | 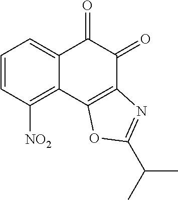 [SMILES: CC(C)c1nc2c(o1)-c1c(cccc1[N+](=O)[O-])C(=O)C2=O] |
| 79 | 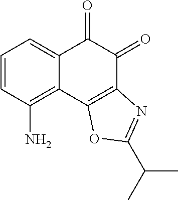 [SMILES: CC(C)c1nc2c(o1)-c1c(N)cccc1C(=O)C2=O] |
| 80 | 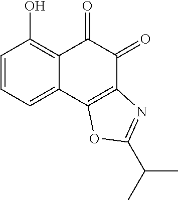 [SMILES: CC(C)c1nc2c(o1)-c1cccc(O)c1C(=O)C2=O] |
| 81 | 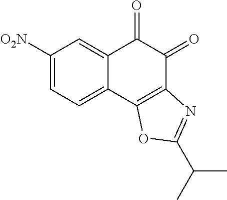 [SMILES: CC(C)c1nc2c(o1)-c1ccc([N+](=O)[O-])cc1C(=O)C2=O] |
| 82 | 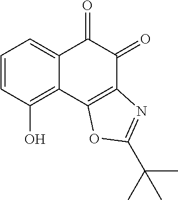 [SMILES: CC(C)(C)c1nc2c(o1)-c1c(O)cccc1C(=O)C2=O] |
| 83 | 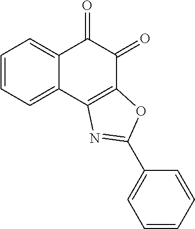 [SMILES: O=C1C(=O)c2oc(-c3ccccc3)nc2-c2ccccc21] |
| 84 | 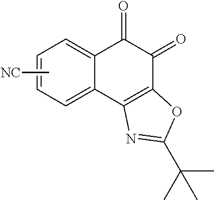 |
| 85 | 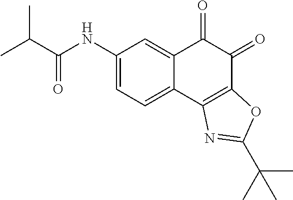 [SMILES: CC(C)C(=O)Nc1ccc2c(c1)C(=O)C(=O)c1oc(C(C)(C)C)nc1-2] |
| 86 | 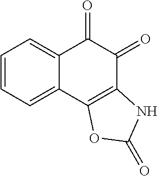 [SMILES: O=C1C(=O)c2[nH]c(=O)oc2-c2ccccc21] |
| 87 | 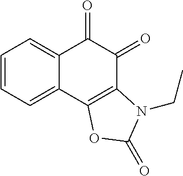 [SMILES: CCn1c2c(oc1=O)-c1ccccc1C(=O)C2=O] |
| 88 | 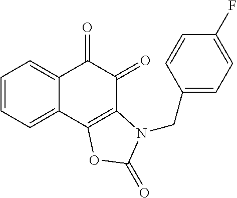 [SMILES: O=C1C(=O)c2c(oc(=O)n2Cc2ccc(F)cc2)-c2ccccc21] |
| 89 | 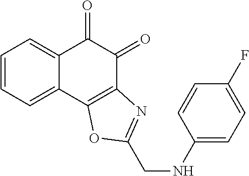 [SMILES: O=C1C(=O)c2nc(CNc3ccc(F)cc3)oc2-c2ccccc21] |
| 90 | 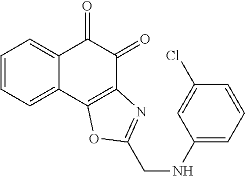 [SMILES: O=C1C(=O)c2nc(CNc3cccc(Cl)c3)oc2-c2ccccc21] |
| 91 | 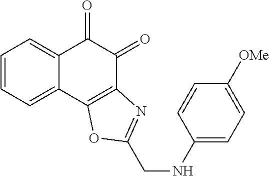 [SMILES: COc1ccc(NCc2nc3c(o2)-c2ccccc2C(=O)C3=O)cc1] |
| 92 | 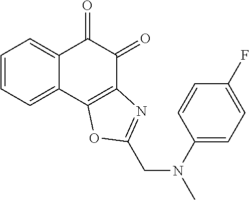 [SMILES: CN(Cc1nc2c(o1)-c1ccccc1C(=O)C2=O)c1ccc(F)cc1] |
| 93 | 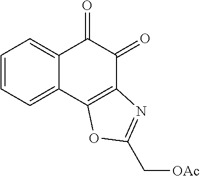 [SMILES: CC(=O)OCc1nc2c(o1)-c1ccccc1C(=O)C2=O] |
| 94 | 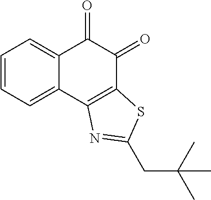 [SMILES: CC(C)(C)Cc1nc2c(s1)C(=O)C(=O)c1ccccc1-2] |
| 95 | 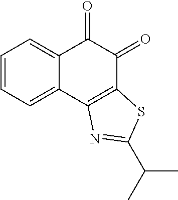 [SMILES: CC(C)c1nc2c(s1)C(=O)C(=O)c1ccccc1-2] |
| 96 | 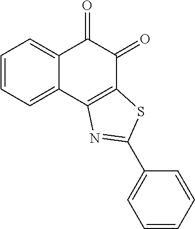 [SMILES: O=C1C(=O)c2sc(-c3ccccc3)nc2-c2ccccc21] |
| 97 | 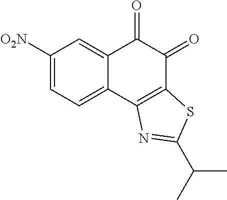 [SMILES: CC(C)c1nc2c(s1)C(=O)C(=O)c1cc([N+](=O)[O-])ccc1-2] |
| 98 | 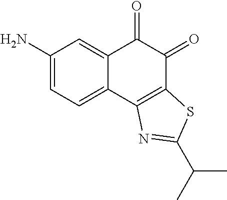 [SMILES: CC(C)c1nc2c(s1)C(=O)C(=O)c1cc(N)ccc1-2] |
| 99 | 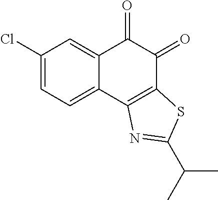 [SMILES: CC(C)c1nc2c(s1)C(=O)C(=O)c1cc(Cl)ccc1-2] |
| 100 | 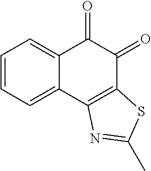 [SMILES: Cc1nc2c(s1)C(=O)C(=O)c1ccccc1-2] |
| 101 | 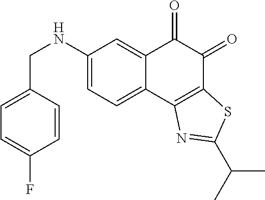 [SMILES: CC(C)c1nc2c(s1)C(=O)C(=O)c1cc(NCc3ccc(F)cc3)ccc1-2] |
| 102 | 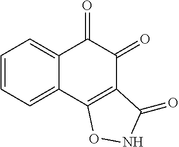 [SMILES: O=C1C(=O)c2c(o[nH]c2=O)-c2ccccc21] |
| 103 | 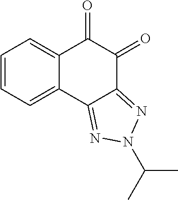 [SMILES: CC(C)n1nc2c(n1)-c1ccccc1C(=O)C2=O] |
| 104 | 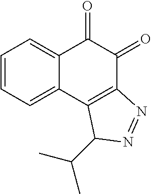 [SMILES: CC(C)C1N=NC2=C1c1ccccc1C(=O)C2=O] |
| 105 | 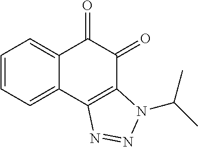 [SMILES: CC(C)n1nnc2c1C(=O)C(=O)c1ccccc1-2] |
| 106 | 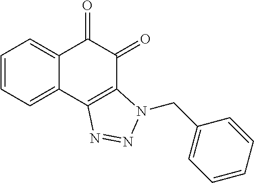 [SMILES: O=C1C(=O)c2c(nnn2Cc2ccccc2)-c2ccccc21] |
| 107 | 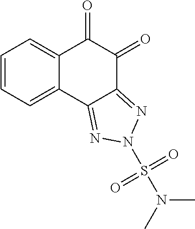 [SMILES: CN(C)S(=O)(=O)n1nc2c(n1)-c1ccccc1C(=O)C2=O] |
| 108 | 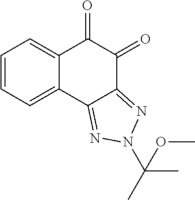 [SMILES: COC(C)(C)n1nc2c(n1)-c1ccccc1C(=O)C2=O] |
| 109 | 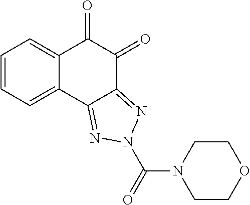 [SMILES: O=C1C(=O)c2nn(C(=O)N3CCOCC3)nc2-c2ccccc21] |
| 110 | 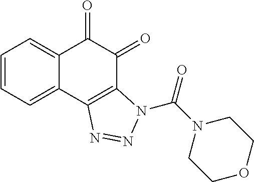 [SMILES: O=C1C(=O)c2c(nnn2C(=O)N2CCOCC2)-c2ccccc21] |
| 111 | 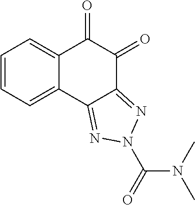 [SMILES: CN(C)C(=O)n1nc2c(n1)-c1ccccc1C(=O)C2=O] |
| 112 | 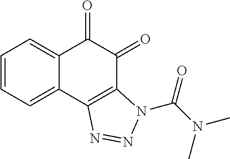 [SMILES: CN(C)C(=O)n1nnc2c1C(=O)C(=O)c1ccccc1-2] |
| 113 | 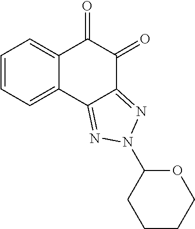 [SMILES: O=C1C(=O)c2nn(C3CCCCO3)nc2-c2ccccc21] |
| 114 | 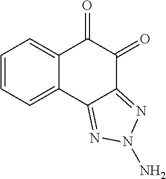 [SMILES: Nn1nc2c(n1)-c1ccccc1C(=O)C2=O] |
| 115 | 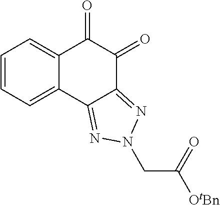 |
| 116 | 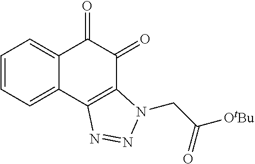 [SMILES: CC(C)(C)OC(=O)Cn1nnc2c1C(=O)C(=O)c1ccccc1-2] |
| 117 | 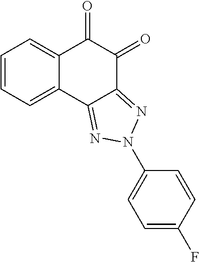 [SMILES: O=C1C(=O)c2nn(-c3ccc(F)cc3)nc2-c2ccccc21] |
| 118 | 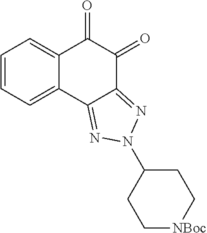 [SMILES: CC(C)(C)OC(=O)N1CCC(n2nc3c(n2)-c2ccccc2C(=O)C3=O)CC1] |
| 119 | 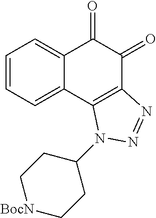 [SMILES: CC(C)(C)OC(=O)N1CCC(n2nnc3c2-c2ccccc2C(=O)C3=O)CC1] |
| 120 | 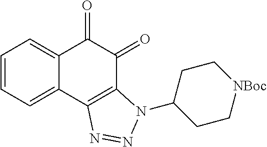 [SMILES: CC(C)(C)OC(=O)N1CCC(n2nnc3c2C(=O)C(=O)c2ccccc2-3)CC1] |
| 121 | 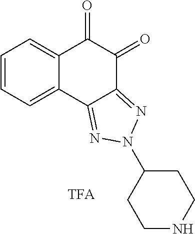 [SMILES: O=C1C(=O)c2nn(C3CCNCC3)nc2-c2ccccc21] |
| 122 | 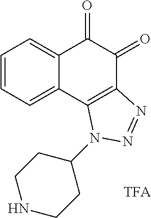 [SMILES: O=C1C(=O)c2nnn(C3CCNCC3)c2-c2ccccc21] |
| 123 | 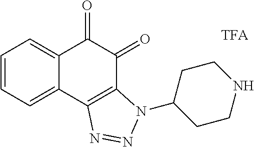 [SMILES: O=C1C(=O)c2c(nnn2C2CCNCC2)-c2ccccc21] |
| 124 | 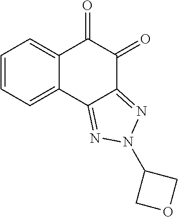 [SMILES: O=C1C(=O)c2nn(C3COC3)nc2-c2ccccc21] |
| 125 | 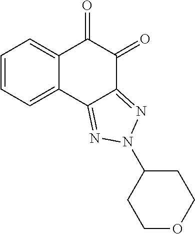 [SMILES: O=C1C(=O)c2nn(C3CCOCC3)nc2-c2ccccc21] |
| 126 | 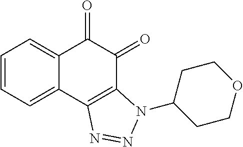 [SMILES: O=C1C(=O)c2c(nnn2C2CCOCC2)-c2ccccc21] |
| 127 | 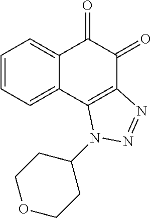 [SMILES: O=C1C(=O)c2nnn(C3CCOCC3)c2-c2ccccc21] |
| 128 | 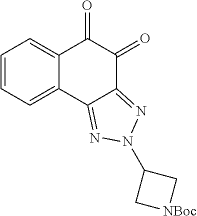 [SMILES: CC(C)(C)OC(=O)N1CC(n2nc3c(n2)-c2ccccc2C(=O)C3=O)C1] |
| 129 | 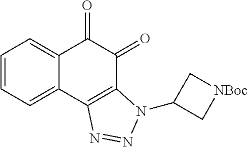 [SMILES: CC(C)(C)OC(=O)N1CC(n2nnc3c2C(=O)C(=O)c2ccccc2-3)C1] |

In addition to the compound of Formula 1, or a pharmaceutically acceptable salt, hydrate, solvate, enantiomer, diasteromer, tautomer or prodrug thereof as an active ingredient, the pharmaceutical composition for preventing or treating an inflammatory disease according to the present invention may further comprise any known drugs used for the prevention or treatment of each type of disease to be treated, any known additives commonly used in the field of the present invention, or the like, and it may be used in combination with other treatments known to treat the disease.

### EXPERIMENTAL EXAMPLES

### Experimental Example 1: In Vitro NQO1 Enzyme Activity Assay

In order to evaluate the enzymatic activities of compounds on NQO1, the experiments were performed as follows:

A compound to measure NQO1 enzyme activity was dissolved in DMSO to prepare a 10 mM stock solution, which was then diluted with DMSO to prepare a working solution at a concentration of 250 μM. For an enzyme reaction solution, 50 μL of 1.54 mM cytochrome C solution was added to 900 μL of 50 mM Tris-HCl (pH 7.5) solution containing 0.14% BSA, and then the prepared working solution at 250 μM was added. After adding 20 μL of 100 ng/ml NQO1 protein, μL of 20 mM NADH solution was added so as to adjust the total volume to 1 mL, and then the change of absorbance was measured at 550 nm for 10 minutes. The change of absorbance was measured at 550 nm for 10 minutes using 1 mL cuvette. The reaction rate was observed through the increase in absorbance as cytochrome C was reduced at 550 nm for 10 minutes, to obtain an absorbance value, and the activity for NQO1 was measured as the reduced amount of cytochrome C (nmol reduced cytochrome C/min/μg NQO1 protein).

Absorption coefficient of Cytochrome C: 21.1 (μmol/mL)-1 cm⁻¹

BSA: Bovine Serum Albumin

Tris-HCl: Tris(hydroxymethyl)aminomethane hydrochloride (buffer solution)

Equipment=Cary 100 UV-Vis Spectrophotometer

The results are shown in Tables 2 and 3.

TABLE 2
NQO1 Activity
(Compound 5 μM, nmol
of reduced C/min/μg
NQO1 Protein)

| | NQO1 2 ng, |
|---|---|
| Cytochrome | Compound |
| Compounds | 5 μM |
| 1 | 180 |
| 3 | 3066 |
| 5 | 2602 |
| 6 | 7706 |

TABLE 3
NQO1 Activity
(Compound 0.2 μM, nmol
of reduced Cytochrome
C/min/μg NQO1 Protein)

| | NQO1 2 ng, |
|---|---|
| | Compound |
| Compounds | 0.2 μM |
| 2 | 3531 |
| 6 | 3782 |
| 7 | 2246 |
| 8 | 3768 |
| 9 | 2474 |
| 10 | 3455 |
| 11 | 2839 |
| 12 | 2981 |
| 13 | 2137 |
| 16 | 4441 |
| 17 | 5843 |
| 18 | 4232 |
| 19 | 4303 |
| 20 | 568 |
| 21 | 1426 |
| 22 | 6978 |
| 23 | 456 |
| 24 | 4336 |
| 25 | 1676 |
| 27 | 2269 |

As shown in Tables 2 to 3, it was observed that the compound of the present invention was used as a substrate of NQO1 and was reduced by receiving electrons from NQO1, and then giving the electrons to cytochrome C.

From the obtained value by measuring the degree of reduction of cytochrome C, it was confirmed that the compound of the present invention has activity in NQO1.

### Experimental Example 2. NAD⁺ Measurement

The primary bone marrow-derived macrophages (BMDMs) isolated from C57BL/6 mouse were incubated in DMEM medium involving macrophage colony-stimulating factor (M-CSF; R&D Systems, 416-ML) during 3 to 5 days. NAD⁺ production and NAD⁺/NADH ratio were measured in isolated BMDMs through using NAD/NADH Assay Kit(ab65348) supplied by Abcam company. All assays were performed according to the manufacturer's product manual.

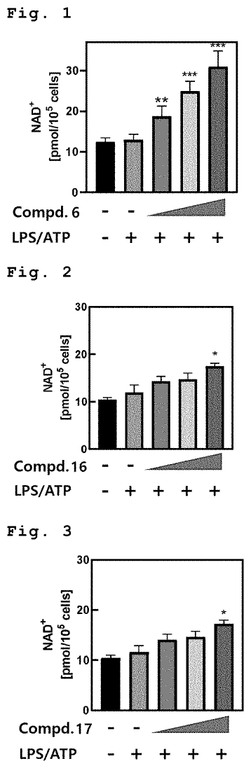

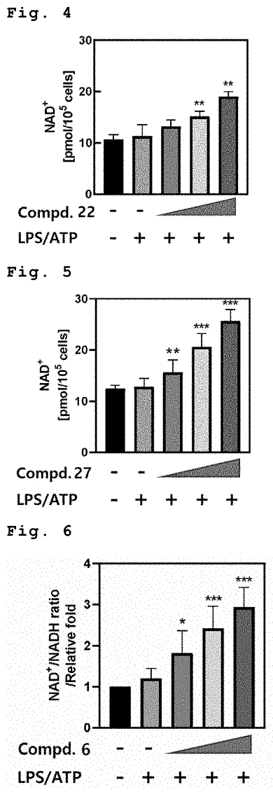

FIGS. 1 to 5 show NAD⁺ changes in the macrophage when treated with compounds 6, 16, 17, 22 and 27 respectively (in order from left in the figure: Untreatment, LPS/ATP, LPS/ATP+1 μM compound, LPS/ATP+5 μM compound, and LPS/ATP+10 μM compound).

As shown in FIGS. 1 to 5, when macrophages were treated with Compounds 6, 16, 17, 22 and 27 respectively after treatment of LPS/ATP, NAD⁺ production was increased in dose-dependent manner due to the reaction of the compound with NQO1 in the cytoplasm.

### Experimental Example 3. NAD⁺/NADH Measurement

The primary bone marrow-derived macrophages (BMDMs) isolated from C57BL/6 mouse were incubated in DMEM medium involving macrophage colony-stimulating factor (M-CSF; R&D Systems, 416-ML) during 3 to 5 days. NAD⁺ production and NAD⁺/NADH ratio was measured in isolated BMDMs through using NAD/NADH Assay Kit(ab65348) supplied by Abcam company. All assays were performed according to the manufacturer's product manual.

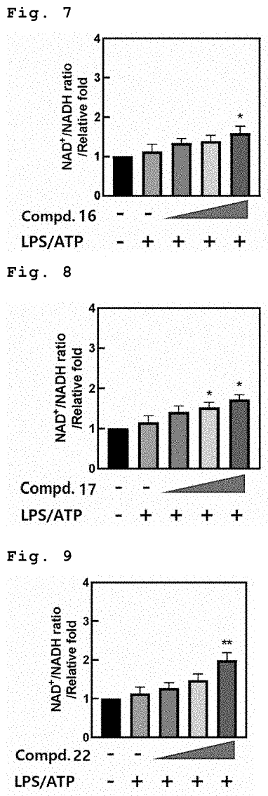

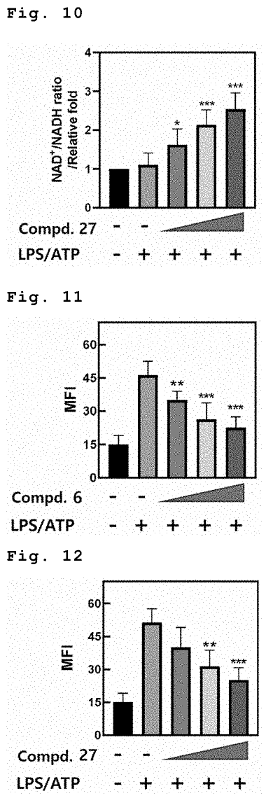

FIGS. 6 to 10 show NAD⁺/NADH ratio changes in the macrophage when treated with compounds 6, 16, 17, and 27 respectively (in order from left in the figure: Untreatment, LPS/ATP, LPS/ATP+1 μM compound, LPS/ATP+5 μM compound, and LPS/ATP+10 μM).

As shown in FIGS. 6 to 10, when macrophages were treated with Compounds 6, 16, 17, 22 and 27 respectively after treatment of LPS/ATP, NAD⁺/NADH ratio was increased in dose-dependent manner due to the reaction of the compound with NQO1 in the cytoplasm.

### Experimental Example 4. Mitochondrial Reactive Oxygen Species Measurement

The primary bone marrow-derived macrophages (BMDMs) isolated from C57BL/6 mouse were incubated in DMEM medium involving macrophage colony-stimulating factor (M-CSF; R&D Systems, 416-ML) during 3 to 5 days. Isolated BMDMs were stained by 1 μM MitoSox (Molecular Probes M36008) for 15 min. After washing the cells into PBS and collecting the cells, mitochondrial reactive oxygen species was measured by FACS.

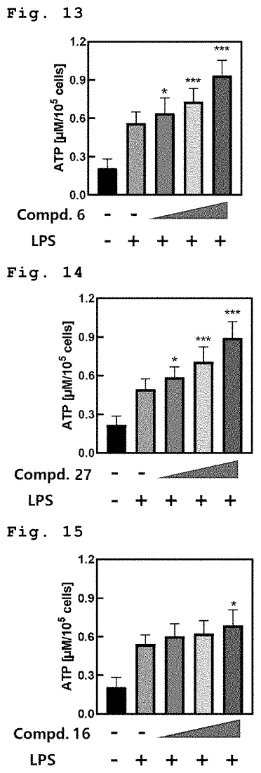

FIGS. 11 and 12 show mitochondrial reactive oxygen species changes in the macrophage when treated with compounds 6 and 27, respectively (in order from left in the figure: Untreatment, LPS/ATP, LPS/ATP+1 μM compound, LPS/ATP+5 μM compound, and LPS/ATP+10 μM compound).

As shown in FIGS. 11 and 12, when treated with Compounds 6 and 27, respectively, in the macrophage after the treatment of LPS/ATP, mitochondrial reactive oxygen species amounts were decreased in dose-dependent manner due to improve mitochondrial function.

### Experiment Example 5. ATP Measurement

The primary bone marrow-derived macrophages (BMDMs) isolated from C57BL/6 mouse were incubated in DMEM medium involving macrophage colony-stimulating factor (M-CSF; R&D Systems, 416-ML) during 3 to 5 days. ATP content in the cell was measured through using ATP colorimetric/fluorometric kit after activation of macrophages by treatment of 100 ng/mL LPS in DMEM medium including 25 FBS. All assays were performed according to the manufacturer's product manual.

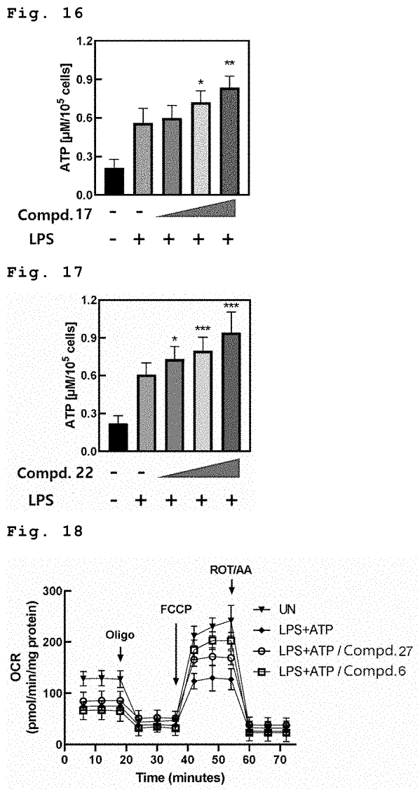

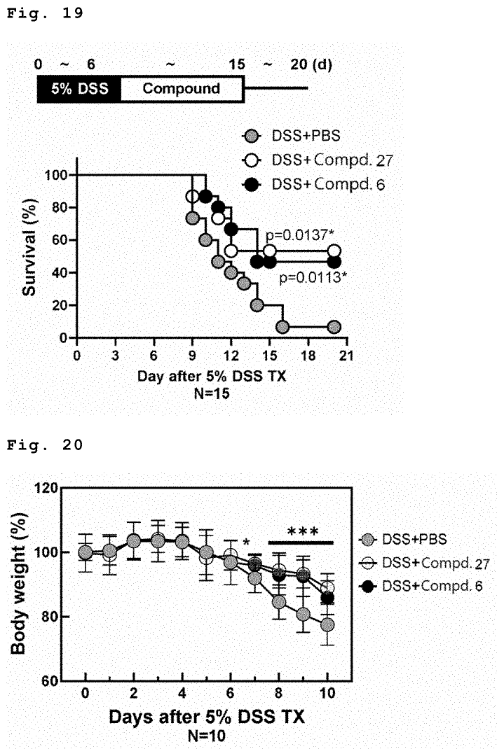

FIGS. 13 to 17 show the measurement of ATP amounts in the macrophage when treated with Compounds 6, 16, 17, 22 and 27 respectively (in order from left in the figure: Untreatment, LPS/ATP, LPS/ATP+1 μM compound, LPS/ATP+5 μM compound, and LPS/ATP+10 μM compound).

As shown in FIGS. 13 and 17, when treated with Compounds 6 and 27, respectively, in the macrophage after treatment of LPS, ATP amounts were increased in dose-dependent manner due to improve mitochondrial function.

### Experimental Example 6. OCR (Oxygen Consumption Rate) Measurement

The primary bone marrow-derived macrophages (BMDMs) isolated from C57BL/6 mouse were incubated in DMEM medium involving macrophage colony-stimulating factor (M-CSF; R&D Systems, 416-ML) during 3 to 5 days. OCR was measured using Seahorse XF24 Extracellular Flux Analyzer in isolated bone marrow-derived macrophages.

FIG. 18 shows the measurement of mitochondrial oxygen consumption rate in the macrophage after the treatment of Compounds 6 and 27, respectively (abbreviations in the figure are as follows: Oligo: Oligomycin; ROT: Rotenone, mitochondrial respiratory chain complex 1 inhibitor; AA: Antimycin A, mitochondrial respiratory chain complex 111 inhibitor; and FCCP: p-trifluoromethoxy carbonyl cyanide phenyl hydrazone).

As shown in FIG. 18, when treated with Compounds 6 and 27, respectively, in the macrophage after treatment of LPS and ATP, overall mitochondrial OCR was increased and it means that the mitochondrial function was improved. This is a supplemental result for the increasing of ATP production shown in FIGS. 13 to 17.

### Experiment Example 7. Efficacy in Ulcerative Colitis Mouse Model

DSS-induced acute colitis mouse model was prepared using 6-week-old C57BL/6 female mouse in the following method from Opal S M et al., Jama., 309(11):1154-1162(2013) and Hotchkiss R S et al., Journal of immunology., 176(9):5471-5477(2006).

The clinical score in colitis were measured daily for body weight, rectal bleeding, total bleeding loss, and fecal concentration during colitis induction. Clinical score was measured by skillful researcher who did not know about the treatment group (Huang L, et al., International Immunopharmacology, 28(1):444-449, 2015).

For immunohistochemistry of tissue section, mouse spleen, lung and colon were fixed using 10% formalin and embedded in paraffin. Paraffin sections were cut to a thickness of 4 μm, and H&E(hematoxylin and eosin) staining was performed. The histopathological score was measured in accordance with the criteria described in Osuchowski M F, et al., Journal of Immunology, 177(3):1967-74(2006); and Liu W, et al., Cell Research, 25(6): 691-706(2015).

The sandwich ELISAs and MPO assay were performed according to conventional methods in this field. TNF-α, IL-6, IL-1β, IL-18 ELISA(sandwich ELISAs) were performed using BD OptEIA ELISA Kit and MPO assay was performed using MPO Activity assay kit(ab105136) of Abcam company.

The data obtained from independent experiment (means±SD) was analyzed by using two-tailed Student's t-test. The differences were considered significant in p<0.05. For survival comparison, the results were graphically described and analyzed according to kaplan-Meier survival analysis, that is, the product-limit method, using log-rank(Mantel-Cox) tests (Prism, version 5.0, GraphPad Software).

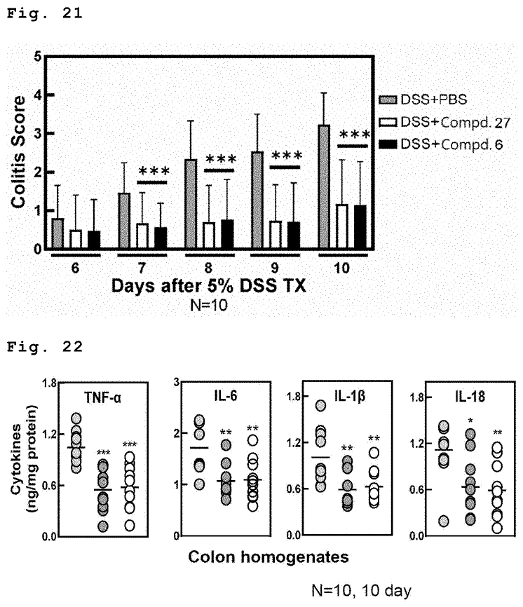

FIG. 19 shows survival rate when treated with Compounds 6 and 27, respectively, in the inflammatory bowel disease mouse model. As shown in FIG. 19, survival rates were higher than non-treated mouse when treated with Compounds 6 and 27, respectively, during 20 days in inflammatory bowel disease mouse model induced by the 5% DSS for 6 days.

FIG. 20 shows body weight changes when treated with Compounds 6 and 27, respectively, in the inflammatory bowel disease mouse model. As shown in FIG. 20, the degree of body weight loss was significantly slower than non-treated mouse when treated with Compounds 27 and 6, respectively, during 10 days in inflammatory bowel disease mouse model induced by the 5% DSS for 6 days.

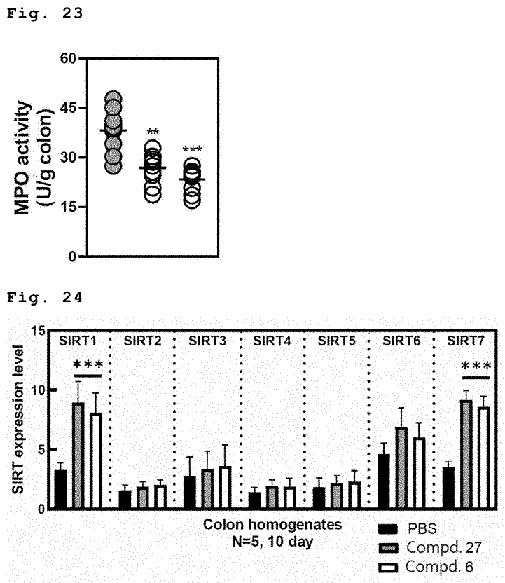

FIG. 21 shows colitis clinic score changes in the inflammatory bowel disease mouse model when treated with Compounds 27 and 6, respectively (in order from left in the figure: DSS, DSS+compound 27, and DSS+Compound 6). As shown in FIG. 21, colitis clinic scores were significantly ameliorated compared with non-treated mouse in the combined score of all score for reduction in body weight (0 point: normal, 1 point: below 5% in reduction of body weight, 2 point: 6-10% in reduction of body weight, 3 point: 11-20% in reduction of body weight, 4 point: above 20% in reduction of body weight), for stool consistency (0 point: normal, 1 point: soft stools, 2 point: very soft stool, 3 point: diarrhea in half of stool, 4 point: diarrhea), and for bleeding in stool or rectal bleeding (0 point: No trace of bleeding, 2 point: bleeding in stool, 4 point: rectal bleeding) when treated with Compounds and 27, respectively, during 10 days in inflammatory bowel disease mouse model induced by the 5% DSS for 6 days.

FIG. 22 shows the results of measurement for cytokine secretions involving INF-α, IL-6, IL-1β and IL-18 using ELISA kit in the centrifuging upper layer of the homogenizing colon tissue with homogenizer in a PBS buffer solution when treated with Compounds 27 and 6, respectively, during 20 days in inflammatory bowel disease mouse model induced by the 5% DSS for 6 days (in order from left in the figure: DSS, DSS+Compound 27, and DSS+Compound 6). As shown in FIG. 22, cytokine expressions were decreased when compound was treated.

FIG. 23 shows the result of measurement for MPO activity using MPO activity analysis kit (ab105136) in the centrifuging upper layer of the homogenizing colon tissue with homogenizer in a PBS buffer solution when treated with Compounds 27 and 6, respectively during 20 days in inflammatory bowel disease mouse model induced by the 5% DSS for 6 days (in order from left in the figure: DSS, DSS+Compound 27, and DSS+Compound 6). As shown in FIG. 23, MPO activity was decreased when compound was treated. 1 unit of MPO activity represents the activity of an enzyme that decomposes H₂O₂ of 1 μmol/min at 25° C. and the activity was described as MPO unit/g colon.

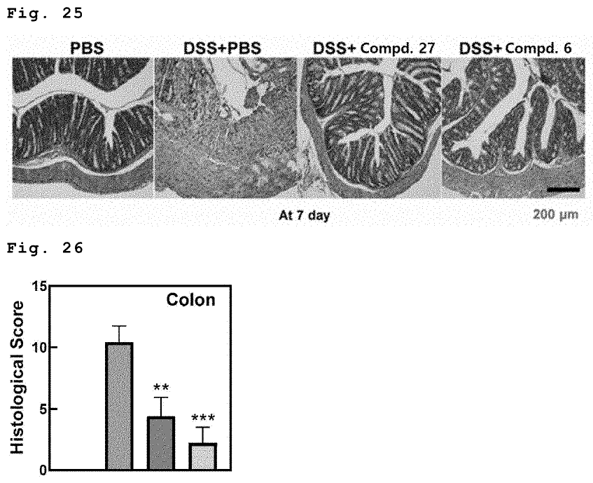

FIG. 24 shows the measured results of a degree of sirtuin expression in the centrifuging upper layer of the homogenizing colon tissue with homogenizer in a PBS buffer solution when treated with Compounds 27 and 6, respectively during 20 days in inflammatory bowel disease mouse model induced by the 5% DSS for 6 days (in order from left in the figure: DSS, DSS+Compound 27, and DSS+Compound 6). As shown in FIG. 24, the expressions of sirtuin 1 and sirtuin 7 were significantly increased when treated with the compound.

FIGS. 25 and 26 show the measured results of immunohistochemistry and histological score in inflammatory bowel disease mouse model after treatment of Compounds 27 and 6, respectively.

After treatment with Compounds 27 and 6, respectively, during 7 days in inflammatory bowel disease mouse model induced by the 5% DSS for 6 days, the isolated colon tissue was fixed with 10% formalin, and it was proceeded for embedding into paraffin, and then cut to observe (FIG. 25), and the scores were averaged by converting the degree of inflammation, its range and tissue damage thought H&E(hematoxylin and eosin) staining (in order from left in the figure: DSS, DSS+compound 27, and DSS+Compound 6). As shown in FIGS. 25 and 26, the colon tissues treated with the compound were significantly improved than non-treated colon tissue in the histological finding.

1. A method for preventing or treating an inflammatory disease comprising administering to a subject in need thereof a pharmaceutical composition comprising an effective amount of a compound of Formula 1, or a pharmaceutically acceptable salt, a hydrate, a solvate, an enantiomer, a diasteromer, a tautomer or a prodrug thereof: 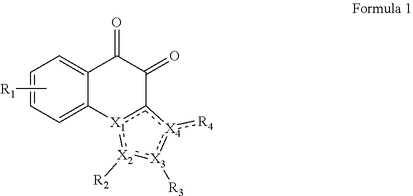
wherein
X₁, X₂, X₃ and X₄ are each independently selected from the group consisting of carbon, nitrogen and sulfur atoms, wherein at least two of X₁, X₂, X₃ and X₄ are hetero atoms selected from nitrogen, oxygen and sulfur, provided that X₁ and X₄ cannot simultaneously be a nitrogen atom;
R₁ is one or more selected from the group consisting of H, alkyl, alkyloxy, C₆₋₁₀ aryl, heteroaryl, halo, nitro, hydroxy, cyano and —NR₅R₆;
R₂ is not present, or selected from the group consisting of H, O, alkyl, alkyloxy, C₆₋₁₀ aryl and heterocyclyl, wherein the alkyl may be substituted with C₆₋₁₀ aryl and the heterocyclyl may be substituted with —C(O)R₈;
R₃ is not present, or selected from the group consisting of H, O, halo, alkyl, alkyloxy, C₆₋₁₀ aryl, heterocyclyl, —SO₂NR₇R₁₂, —NR₉R₁₀ and —C(O)R₁₁, wherein when the alkyl is substituted, its substituent is selected from the group consisting of halo, alkyloxy, C₆₋₁₀ aryl, C₆₋₁₀ aryloxy, heterocyclyl, —C(O)R₈, R₁₂C(O)O— and —NR₁₃R₁₄, and the heterocyclyl may be substituted with —C(O)R₈;
R₄ is not present, or selected from the group consisting of H, O, alkyl, alkyloxy, C₆₋₁₀ aryl, C₆₋₁₀ aryloxy, heterocyclyl and —C(O)R₁₅, wherein when the alkyl is substituted, its substituent is selected from the group consisting of halo, C₆₋₁₀ aryl, heterocyclyl and —C(O)R₈, and the heterocyclyl may be substituted with —C(O)R₈;
R₅ and R₆ are each independently selected from the group consisting of H, alkyl and —C(O)R₇, or R₅ and R₆ are joined with each other to form a heterocyclyl including at least one nitrogen atom in the cycle;
R₇ and R₁₂ are each alkyl, or R₇ and R₁₂ are joined with each other to form a heterocyclyl including at least one nitrogen atom in the cycle;
R₁₁ is heterocyclyl or —NR₁₃R₁₄;
R₁₅ is alkyl, alkyloxy, C₆₋₁₀ aryloxy, heterocyclyl or —NR₁₃R₁₄, R₉, R₁₀, R₁₃ and R₁₄ are each independently selected from the group consisting of H, alkyl, unsubstituted or halo-substituted C₆₋₁₀ aryl, and —C(O)R₈, or either R₉ and R₁₀ are jointed with each other, or R₁₃ and R₁₄ are jointed with each other, to form a heterocyclyl including at least one nitrogen atom in the cycle;
R₈ is alkyloxy;
the alkyl is each C₆₋₁₀ linear or branched alkyl, or C₃₋₇ cyclic alkyl, the heterocyclyl is 3- to 7-membered heterocyclic group having in the cycle at least one hetero atom selected from the group consisting of N, O and S, the heteroaryl is 5- to 10-membered aromatic cyclic group having in the cycle at least one hetero atom selected from N, O and S, and when the aryl or heteroaryl is substituted, its substituent is each at least one selected from the group consisting of halo, alkyl, halo-substituted alkyl and alkyloxy; and [symbol]
is a single bond or a double bond depending on R₂, R₃, R₄, X₁, X₂, X₃ and X₄,
provided that when both X₁ and X₄ are carbon atom, and both X₂ and X₃ are nitrogen atom, either of R₂ or R₄ is not alkyl, aryl or heterocyclyl, and herein, when R₂ is alkyl, aryl or heterocyclyl, R₄ is not —C(O)R₁₅; and when both X₁ and X₄ are carbon atom, and both X₃ and X₄ are nitrogen atom, either of R₂ or R₄ is O or alkyloxy.

2. The method according to claim 1, wherein
both X₁ and X₄ are carbon atoms, and both X₂ and X₃ are nitrogen atoms,
R₂ is not present, or alkyl, alkyloxy or C₆₋₁₀ aryl,
R₃ is not present, or H, alkyl or C₆₋₁₀ aryl, and
R₄ is not present, or O, alkyl or alkyloxy,
provided that either R₂ or R₄ is not alkyl, aryl or heterocyclyl, herein, when R₂ is alkyl, aryl or heterocyclyl, R₄ is not —C(O)R₁₅.

3. The method according to claim 1, wherein
both X₁ and X₃ are carbon atoms, and both X₂ and X₄ are nitrogen atoms,
R₂ is not present or alkyl,
R₃ is selected from the group consisting of halo, alkyl, alkyloxy, C₆₋₁₀ aryl and heterocyclyl,
R₄ is not present, or selected from the group consisting of H, alkyl, C₆₋₁₀ aryl, C₆₋₁₀ aryloxy, heterocyclyl and —C(O)R₁₅,
R₁₅ is alkyl, alkyloxy, C₆₋₁₀ aryloxy, heterocyclyl or —NR₁₃R₁₄,
R₁₃ and R₁₄ are each independently selected from the group consisting of H, alkyl, unsubstituted or halo-substituted C₆₋₁₀ aryl, and —C(O)R₈, and
R₈ is alkyloxy.

4. The method according to claim 1, wherein
both X₁ and X₃ are carbon atoms, X₂ is nitrogen atom, X₄ is sulfur atom, R₂ and R₄ are not present, and R₃ is alkyl or C₆₋₁₀ aryl.

5. The method according to claim 1, wherein
both X₁ and X₃ are carbon atoms, one of X₂ and X₄ is nitrogen atom and the other is oxygen atom, R₂ is not present, R₃ is O, alkyl or C₆₋₁₀ aryl, and R₄ is not present, or H or alkyl.

6. The method according to claim 1, wherein
X₂, X₃ and X₄ are nitrogen atoms,
R₂ is not present, alkyl or heterocyclyl,
R₃ is not present, or selected from the group consisting of alkyl, C₆₋₁₀ aryl, heterocyclyl, —SO₂R₇R₁₂, —NR₉R₁₀ and —C(O)R₁₁,
R₄ is not present, or selected from the group consisting of alkyl, heterocyclyl and —C(O)R₁₅,
R₇ and R₁₂ are each independently alkyl,
R₁₁ is heterocyclyl or —NR₁₃R₁₄,
R₁₅ is alkyl, alkyloxy, C₆₋₁₀ aryloxy, heterocyclyl or —NR₁₃R₁₄,
R₉, R₁₀, R₁₃ and R₁₄ are each independently selected from the group consisting of H, alkyl, unsubstituted or halo-substituted C₆₋₁₀ aryl, and —C(O)R₈, and
R₈ is alkyloxy.

7. The method according to claim 1, wherein
both X₂ and X₃ are carbon atoms, both X₁ and X₄ are nitrogen atoms, R₂ is C₆₋₁₀ aryl, and R₃ and R₄ are not present.

8. The method according to claim 1, wherein the compound of Formula 1 is selected from the group consisting of the following:

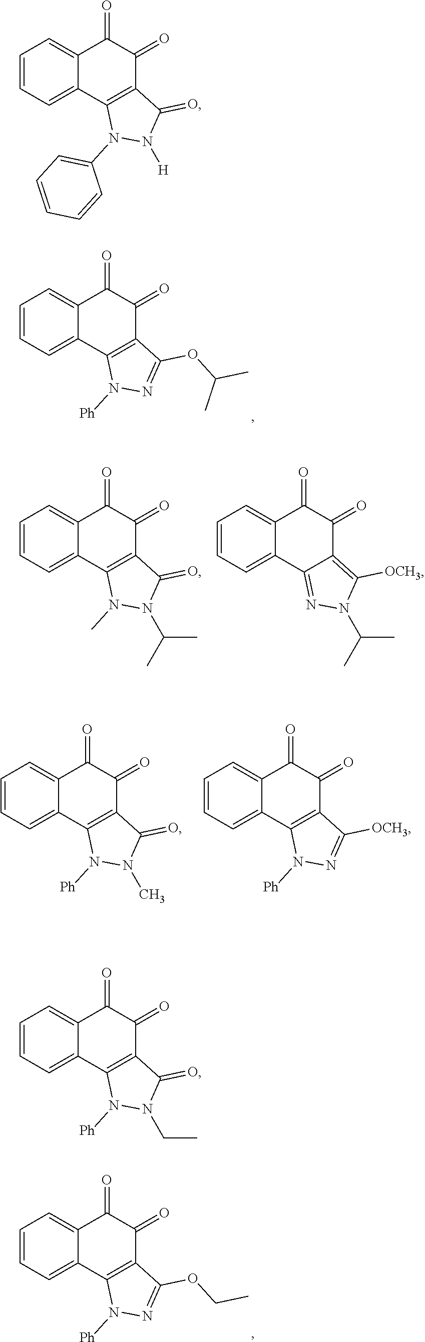

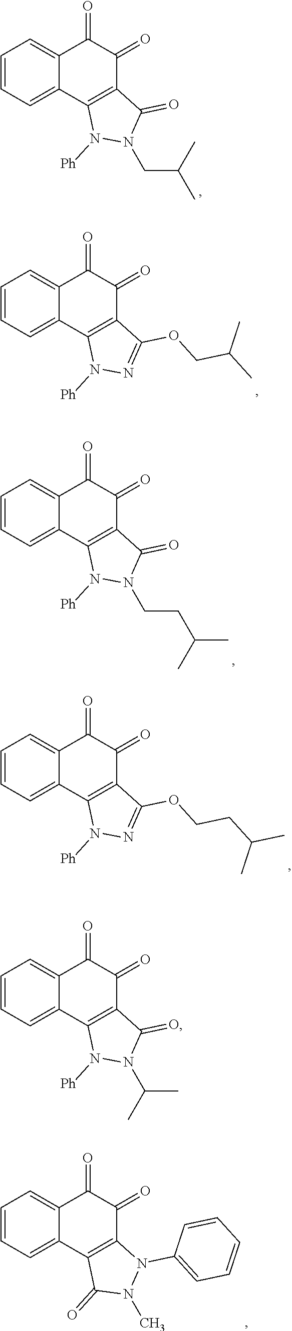 [SMILES: CC(C)Cn1c(=O)c2c(n1-c1ccccc1)-c1ccccc1C(=O)C2=O ; CC(C)COc1nn(-c2ccccc2)c2c1C(=O)C(=O)c1ccccc1-2 ; CC(C)CCn1c(=O)c2c(n1-c1ccccc1)-c1ccccc1C(=O)C2=O ; CC(C)CCOc1nn(-c2ccccc2)c2c1C(=O)C(=O)c1ccccc1-2 ; CC(C)n1c(=O)c2c(n1-c1ccccc1)-c1ccccc1C(=O)C2=O ; Cn1c(=O)c2c(n1-c1ccccc1)C(=O)C(=O)c1ccccc1-2]

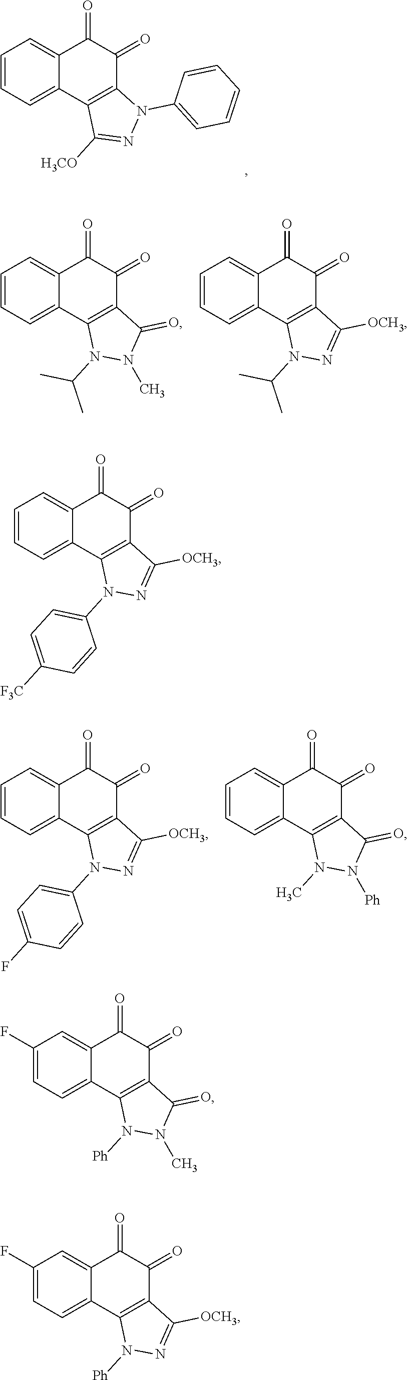

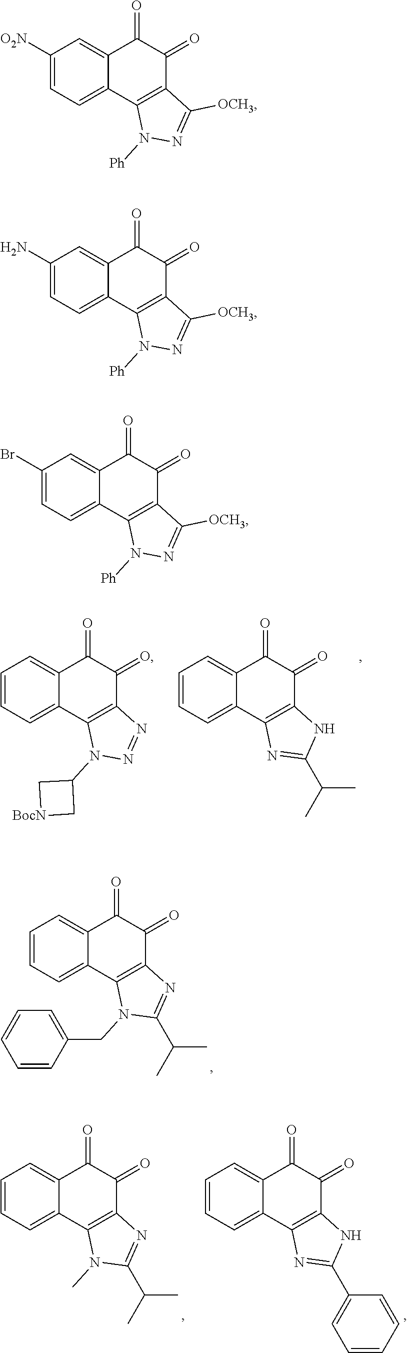

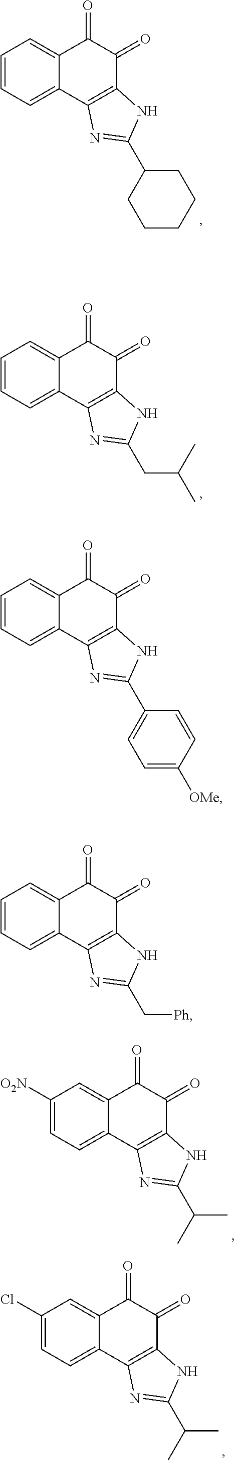

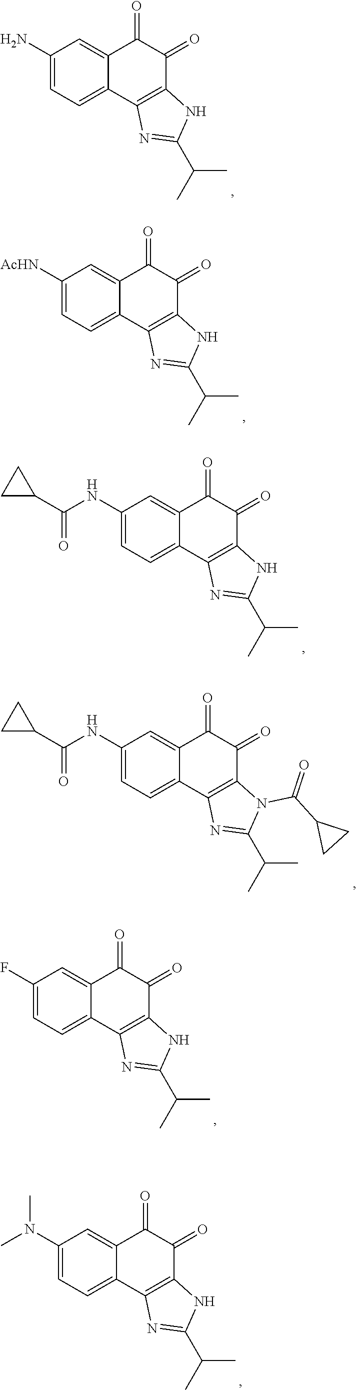

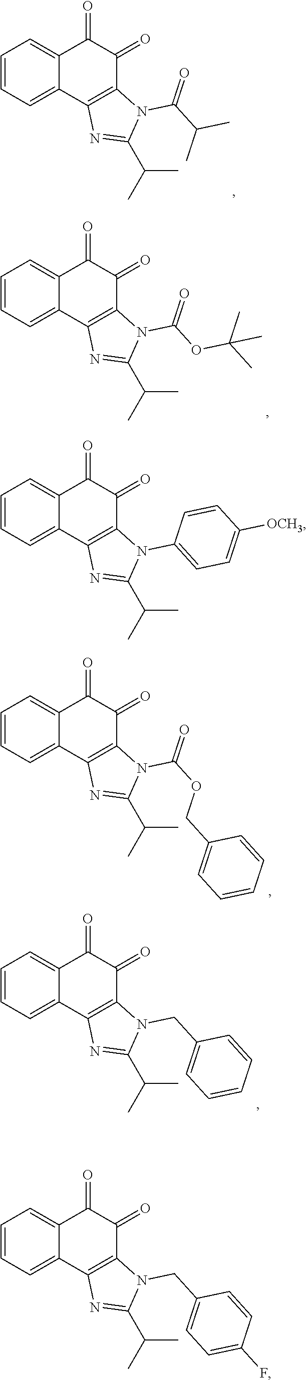

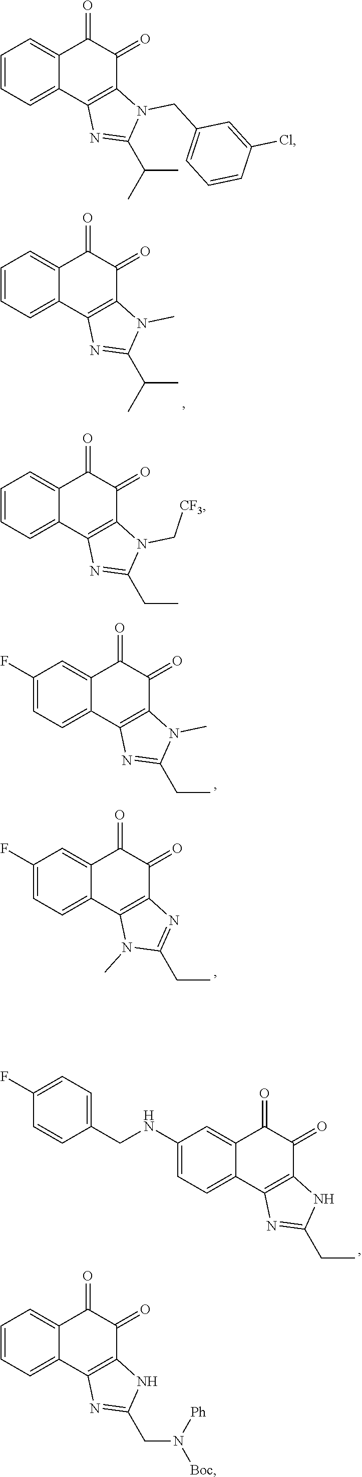

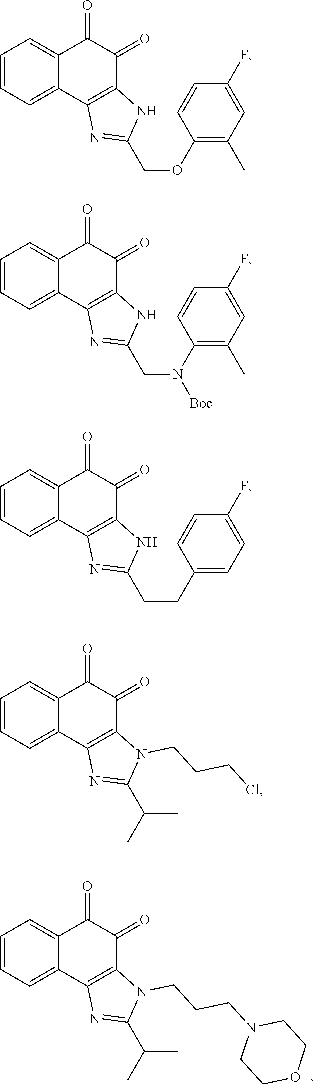 [SMILES: Cc1cc(F)ccc1OCc1nc2c([nH]1)C(=O)C(=O)c1ccccc1-2 ; Cc1cc(F)ccc1N(Cc1nc2c([nH]1)C(=O)C(=O)c1ccccc1-2)C(=O)OC(C)(C)C ; O=C1C(=O)c2[nH]c(CCc3ccc(F)cc3)nc2-c2ccccc21 ; CC(C)c1nc2c(n1CCCCl)C(=O)C(=O)c1ccccc1-2 ; CC(C)c1nc2c(n1CCCN1CCOCC1)C(=O)C(=O)c1ccccc1-2]

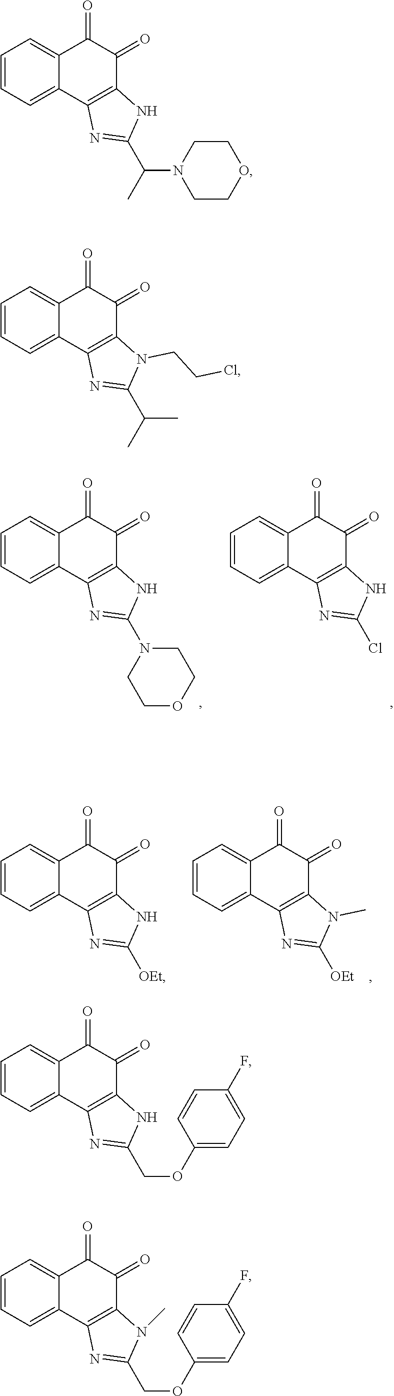

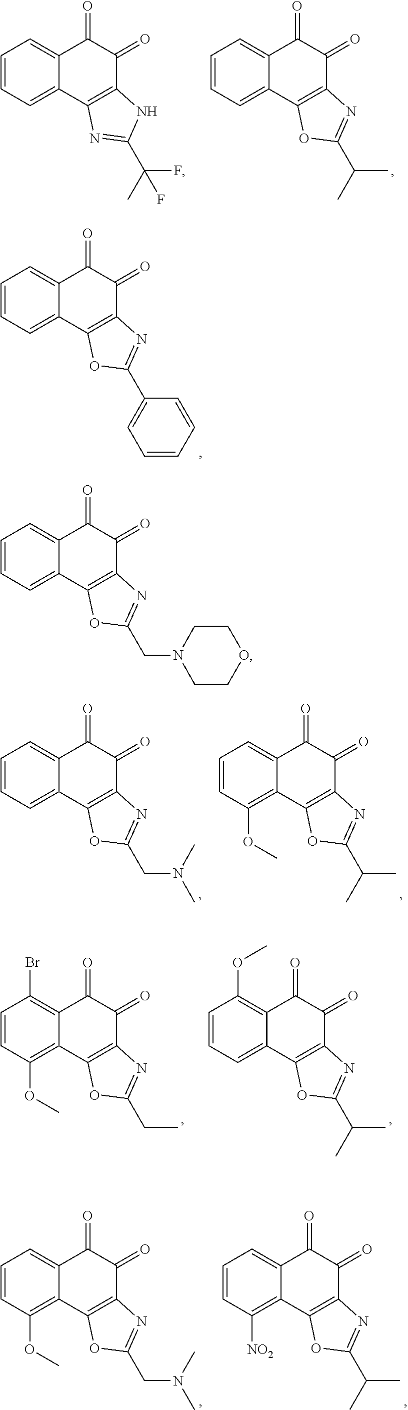

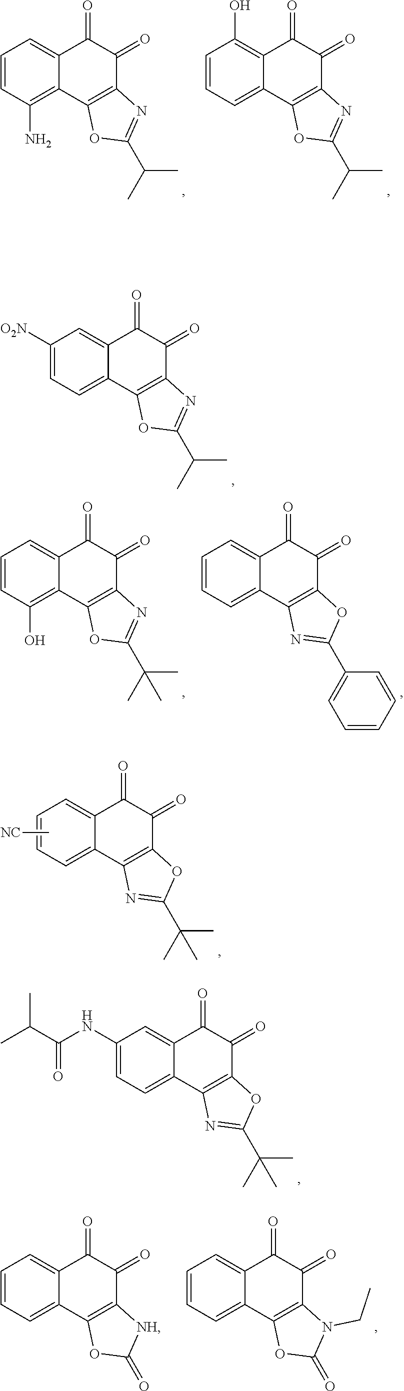

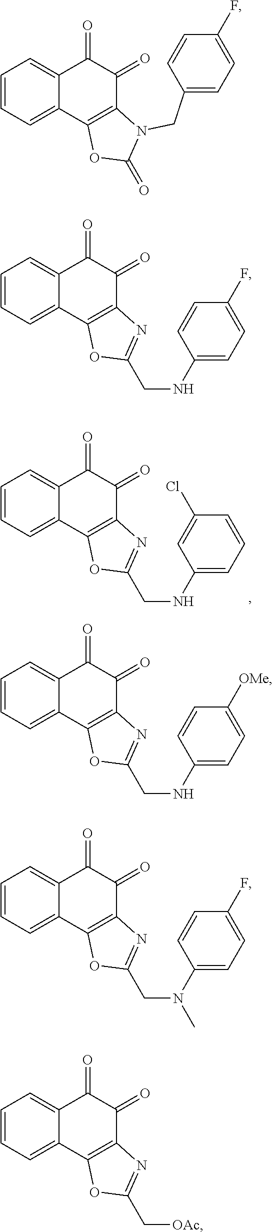 [SMILES: O=C1C(=O)c2c(oc(=O)n2Cc2ccc(F)cc2)-c2ccccc21 ; O=C1C(=O)c2nc(CNc3ccc(F)cc3)oc2-c2ccccc21 ; O=C1C(=O)c2nc(CNc3cccc(Cl)c3)oc2-c2ccccc21 ; COc1ccc(NCc2nc3c(o2)-c2ccccc2C(=O)C3=O)cc1 ; CN(Cc1nc2c(o1)-c1ccccc1C(=O)C2=O)c1ccc(F)cc1 ; CC(=O)OCc1nc2c(o1)-c1ccccc1C(=O)C2=O]

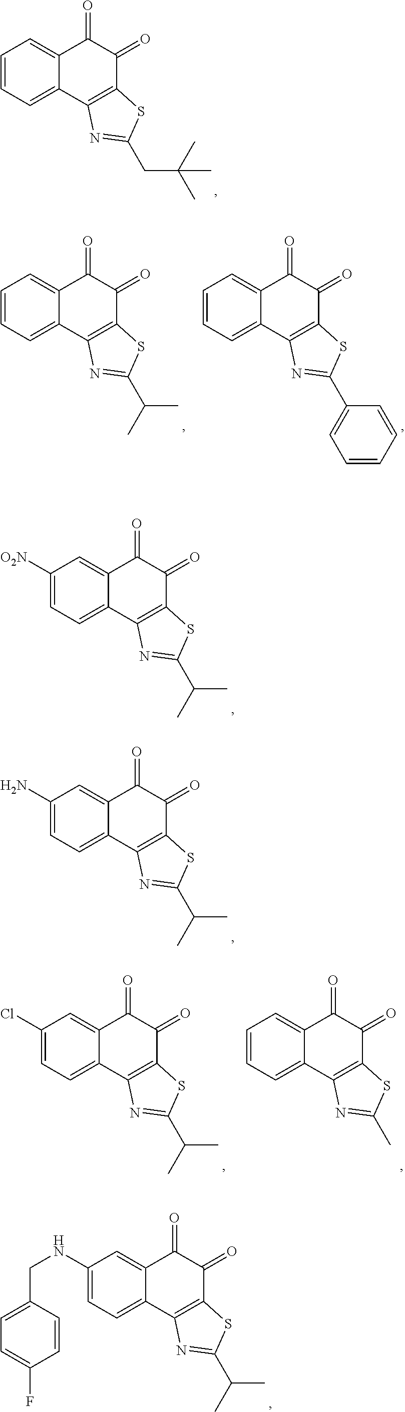

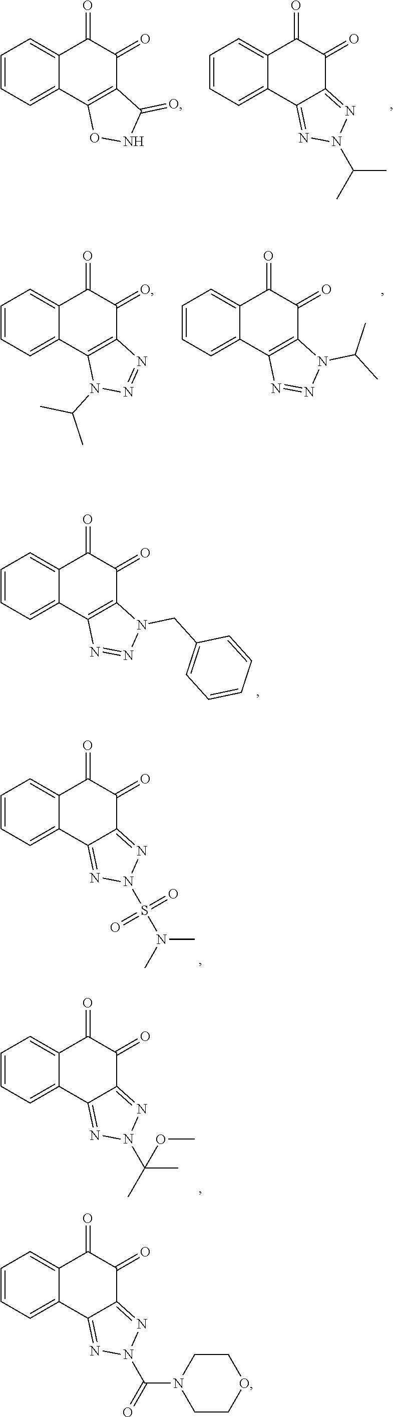

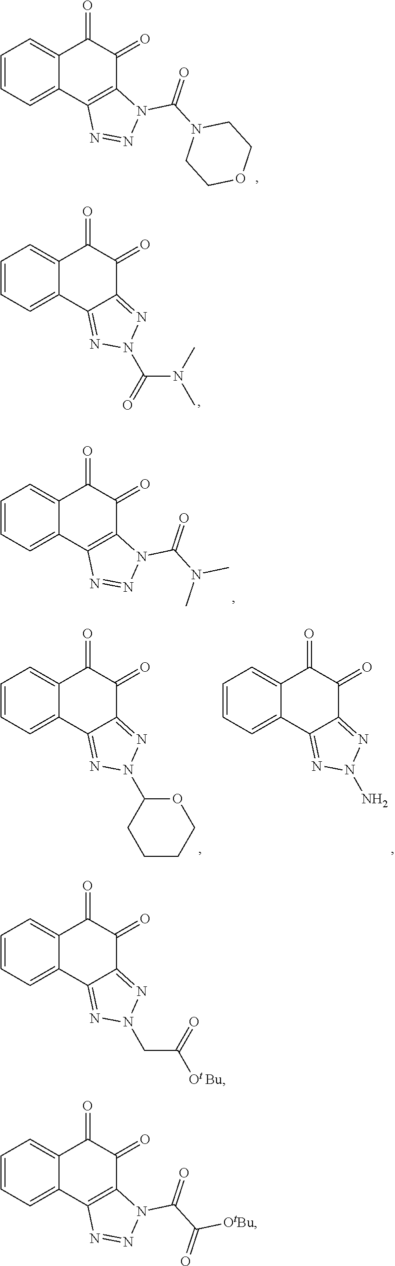

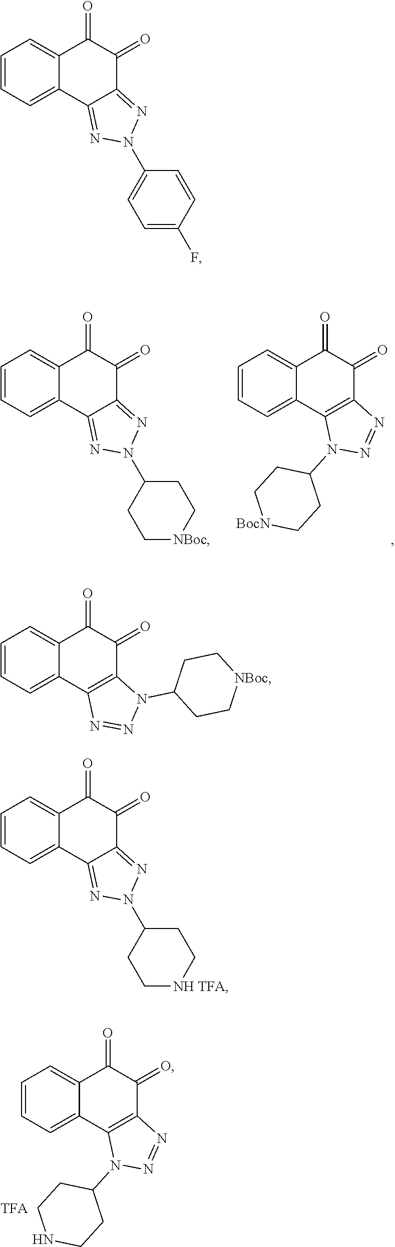

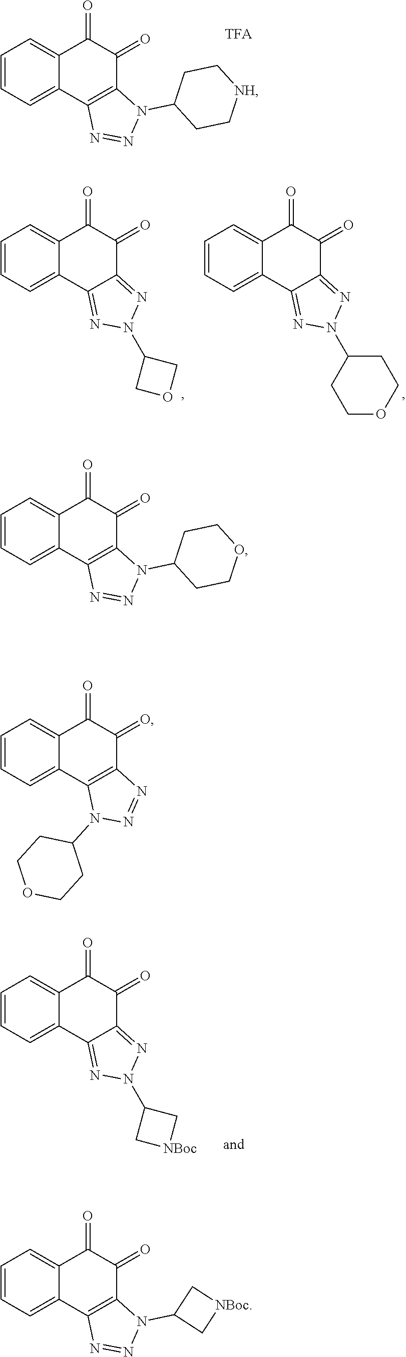

9. The method according to claim 1, wherein the inflammatory disease is selected from the group consisting of ulcerative colitis, sepsis, rheumatoid arthritis, multiple sclerosis, Crohn's disease and atopic dermatitis.
